SEQUENCE LISTING (24 sequences)

SEQ ID NO: 1 - protein, 8 aa, synthetic construct
  REGION 1..8: note = consensus sequence
  VARIANT 3: note = Xaa can be any naturally occurring amino acid
  VARIANT 6..7: note = Xaa can be any naturally occurring amino acid
LDXLLXXL

SEQ ID NO: 2 - DNA, 22 bp, synthetic construct
  misc_feature 1..22: note = upstream USP1R primer
  source 1..22: mol_type = other DNA
aggttcacagcattctcaatcc

SEQ ID NO: 3 - DNA, 21 bp, synthetic construct
  misc_feature 1..21: note = upstream USP1F primer
  source 1..21: mol_type = other DNA
cagtgcctgtgaaactttgga

SEQ ID NO: 4 - DNA, 20 bp, synthetic construct
  misc_feature 1..20: note = promoter USP1F primer
  source 1..20: mol_type = other DNA
ctcagctctacagcattcgc

SEQ ID NO: 5 - DNA, 20 bp, synthetic construct
  misc_feature 1..20: note = promoter USP1R primer
  source 1..20: mol_type = other DNA
ggccatccaatgagacaagg

SEQ ID NO: 6 - DNA, 21 bp, synthetic construct
  misc_feature 1..21: note = PXN siRNA
  source 1..21: mol_type = other DNA
gcttcgctgtcggatttcatt

SEQ ID NO: 7 - DNA, 25 bp, synthetic construct
  misc_feature 1..25: note = ITGB4 siRNA
  source 1..25: mol_type = other DNA
cagttctgcgagtatgacaacttcc

SEQ ID NO: 8 - DNA, 25 bp, synthetic construct
  misc_feature 1..25: note = ITGB4 siRNA
  source 1..25: mol_type = other DNA
cgagaagcttcacacctatttccct

SEQ ID NO: 9 - DNA, 25 bp, synthetic construct
  misc_feature 1..25: note = ITGB4 siRNA
  source 1..25: mol_type = other DNA
gtcagttctgcgagtatgacaactt

SEQ ID NO: 10 - DNA, 25 bp, synthetic construct
  misc_feature 1..25: note = USP1 siRNA
  source 1..25: mol_type = other DNA
agctacaagtgatacattagagagt

SEQ ID NO: 11 - RNA, 25 nt, synthetic construct
  misc_feature 1..25: note = USP1 siRNA
  source 1..25: mol_type = other RNA
ggagcacaaagccaactaacgarca

SEQ ID NO: 12 - DNA, 25 bp, synthetic construct
  misc_feature 1..25: note = USP1 siRNA
  source 1..25: mol_type = other DNA
ggcaagttatgaattgatatgcagt

SEQ ID NO: 13 - RNA, 25 nt, synthetic construct
  misc_feature 1..25: note = VDAC1 siRNA
  source 1..25: mol_type = other RNA
acaacactcagaatctaaattggac

SEQ ID NO: 14 - DNA, 25 bp, synthetic construct
  misc_feature 1..25: note = VDAC1 siRNA
  source 1..25: mol_type = other DNA
ggaatttcaagcataaatgaatact

SEQ ID NO: 15 - RNA, 25 nt, synthetic construct
  misc_feature 1..25: note = VDAC1 siRNA
  source 1..25: mol_type = other RNA
gcaccagagtatgaaatagcttcca

SEQ ID NO: 16 - DNA, 25 bp, synthetic construct
  misc_feature 1..25: note = ITGA7 siRNA
  source 1..25: mol_type = other DNA
agtgaagtcctccataaagaacttg

SEQ ID NO: 17 - RNA, 25 nt, synthetic construct
  misc_feature 1..25: note = ITGA7 siRNA
  source 1..25: mol_type = other RNA
gtatgaggtcacggtttccaaccaa

SEQ ID NO: 18 - DNA, 25 bp, synthetic construct
  misc_feature 1..25: note = ITGA7 siRNA
  source 1..25: mol_type = other DNA
cttagtttgctgccatcagtctagt

SEQ ID NO: 19 - RNA, 25 nt, synthetic construct
  misc_feature 1..25: note = KIF14 siRNA
  source 1..25: mol_type = other RNA
gcactgacaagaaatgttataaaga

SEQ ID NO: 20 - RNA, 25 nt, synthetic construct
  misc_feature 1..25: note = KIF14 siRNA
  source 1..25: mol_type = other RNA
cacgatcacagaataacaagtcgaa

SEQ ID NO: 21 - DNA, 25 bp, synthetic construct
  misc_feature 1..25: note = KIF14 siRNA
  source 1..25: mol_type = other DNA
ggcataactagttgaaattggccat

SEQ ID NO: 22 - DNA, 25 bp, synthetic construct
  misc_feature 1..25: note = G3BP1 siRNA
  source 1..25: mol_type = other DNA
gtgcgagaacaacgaataaatattc

SEQ ID NO: 23 - RNA, 25 nt, synthetic construct
  misc_feature 1..25: note = G3BP1 siRNA
  source 1..25: mol_type = other RNA
cccgtaagaaggaatgttactttaa

SEQ ID NO: 24 - RNA, 25 nt, synthetic construct
  misc_feature 1..25: note = G3BP1 siRNA
  source 1..25: mol_type = other RNA
accacctcatgttgttaaagtacca

(19) United States
(12) Patent Application Publication
(10) Pub. No.: US 2025/0009836 A1
(43) Pub. Date: Jan. 9, 2025

(54) COMPOSITIONS AND METHODS OF ALLEVIATING RESISTANCE TO CHEMOTHERAPY

(71) Applicant: CITY OF HOPE, Duarte, CA (US)

(72) Inventors: Ravi SALGIA, Pasadena, CA (US); Atish MOHANTY, Arcadia, CA (US)

(73) Assignee: CITY OF HOPE, Duarte, CA (US)

(21) Appl. No.: 18/653,869

(22) Filed: May 2, 2024

Related U.S. Application Data
(63) Continuation of application No. 17/029,465, filed on Sep. 23, 2020, now abandoned.
(60) Provisional application No. 62/904,195, filed on Sep. 23, 2019.

(51) Int. Cl. A61K 38/07; A61K 39/395; A61K 45/06; A61P 35/00; C07K 16/28; C12N 15/113

(52) U.S. Cl. CPC A61K 38/07; A61K 39/3955; A61K 45/06; A61P 35/00; C07K 16/28; C07K 16/2839; C12N 15/113; C12N 15/1138; C07K 2317/732; C12N 2310/14

(57) ABSTRACT

Disclosed herein are methods and compositions for alleviating resistance to chemotherapy in a subject using one or more therapeutic agents that perturb the interaction between integrin β4 (ITGB4) and paxillin (PXN), e.g., by inhibiting the expression of ITGB4, PXN, or both. Also disclosed is a combinational therapy for treating cancer with a chemotherapeutic agent such as cisplatin or carboplatin and an inhibitor of ITGB4, PXN or both, such as carfilzomib.

### CROSS-REFERENCE TO RELATED APPLICATIONS

The present application is a continuation of U.S. application Ser. No. 17/029,465, filed Sep. 23, 2020, which claims priority to U.S. Provisional Application No. 62/904,195, filed Sep. 23, 2019, the content of which are incorporated herein by reference in their entirety, including drawings.

### SEQUENCE LISTING

This application contains a ST.26 compliant Sequence Listing, which was submitted in XML format via Patent Center, and is hereby incorporated by reference in its entirety. The XML copy, created on Aug. 7, 2024, is named 0544358198US02.xml and is 32,000 bytes in size.

### BACKGROUND

Lung cancer is the most frequently diagnosed cancer and a leading cause of cancer-related deaths worldwide (Bray et al, 2018). Approximately 85% of patients have a group of histological subtypes collectively known as non-small cell lung cancer (NSCLC), of which lung adenocarcinoma (LUAD) and lung squamous cell carcinoma are the most common subtypes, followed by squamous cell carcinoma and less so, large-cell carcinoma (Salgia, 2016; Herbst et al, 2018). LUAD accounts for ˜40% of all lung cancers. These histologies possess different clinical characteristics, and there are potential differences in response to cytotoxic chemotherapies. Approximately 40-50% of patients with NSCLC will be diagnosed with advanced or metastatic disease and are not candidates for curative therapy. Recent advances have transformed lung cancer care with a percentage of patients receiving first-line tyrosine kinase inhibitors based on their genomic-informed markers (Tan et al, 2017). However, immunotherapy alone or in combination with platinum-based chemotherapy is now the recommended first-line treatment option for the remainder of these patients.

Cisplatin [Cis-diamminedichloroplatinum(II)] is a widely prescribed platinum-based compound that exerts clinical activity against a wide spectrum of solid neoplasms, including testicular, bladder, ovarian, colorectal, lung, and head and neck cancers (Galluzzi et al, 2012; Galluzzi et al, 2014). Cisplatin treatment is generally associated with high rates of clinical responses. However, in the vast majority of cases, malignant cells exposed to cisplatin activate a multipronged adaptive response that renders them less susceptible to the anti-proliferative and cytotoxic effects of the drug, and eventually resume proliferation. Thus, a large number of cisplatin-treated patients experience therapeutic failure and tumor recurrence. However, the exact mechanism(s) underlying the emergence of drug resistance remains poorly understood and a bewildering plethora of targets have been implicated in different cancer types. For example, in NSCLC alone, dysregulation of genes involved in cell cycle arrest and apoptosis namely, mouse double minute 2 homolog (MDM2), xeroderma pigmentosum complementation group C, stress inducible protein and p21 (Sarin et al, 2017), cytoplasmic RAP1 that alters NF-κB signaling, upregulation of antiapoptotic factor BCL-2 (Xiao et al, 2017), enhanced Stat3 and Akt phosphorylation, high expression of survivin (Hu et al, 2016), hypoxia factor HIF-1a and mutant p53 (Deben et al, 2018), have been implicated in cisplatin resistance. Furthermore, although changes in administration schedules, choice of methods, and frequency of toxicity monitoring have all contributed to incremental improvements, chemoresistance limits the clinical utility of cisplatin (Fennell et al, 2016). Therefore, there is a dire need for a deeper understanding of chemoresistance and the identification of prognostic and predictive markers to discern responders from non-responders.

In sum, certain potent chemotherapeutic agents such as cisplatin are used to treat a variety of solid tumors, including lung adenocarcinoma (LUAD). Unfortunately, almost all patients develop resistance to the drug in the long term. Therefore, there is a need in the field to resolve chemoresistance to expand the currently limited therapeutic options.

### SUMMARY

In one aspect, disclosed herein is a method of alleviating resistance to chemotherapy in a subject, the method includes administering to the subject a composition comprising one or more therapeutic agents that perturb the interaction between integrin β4 (ITGB4) and paxillin (PXN), e.g., by inhibiting the expression of ITGB4, PXN, or both. In some embodiments, the one or more therapeutic agents include an anti-ITGB4 antibody, an siRNA that inhibits ITGB4 expression, an anti-PXN antibody, and/or an siRNA that inhibits PXN expression. In some embodiments, the composition comprises one or more compounds selected from carfilzomib, ixazomib, and CUDC-101. In some embodiments, the subject suffers from one or more solid tumors. In some embodiments, the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma. In some embodiments, the subject has developed or is at an elevated risk of developing resistance to platinum-based chemotherapy. In some embodiments, the subject has developed or is at an elevated risk of developing resistance to cisplatin or carboplatin.

In another aspect, disclosed herein is a composition for alleviating resistance to chemotherapy in a subject. The composition comprises one or more therapeutic agents that perturb the interaction between integrin β4 (ITGB4) and paxillin (PXN), e.g., by inhibiting the expression of ITGB4, PXN, or both. In some embodiments, the one or more therapeutic agents include an anti-ITGB4 antibody, an siRNA that inhibits ITGB4 expression, an anti-PXN antibody, and/or an siRNA that inhibits PXN expression. In some embodiments, the composition comprises one or more compounds selected from carfilzomib, ixazomib, and CUDC-101. In some embodiments, the subject suffers from one or more solid tumors. In some embodiments, the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma. In some embodiments, the subject has developed or is at an elevated risk of developing resistance to platinum-based chemotherapy. In some embodiments, the subject has developed or is at an elevated risk of developing resistance to cisplatin or carboplatin.

In a related aspect, disclosed herein is a method of treating cancer in a subject, the method includes administering to the subject a chemotherapeutic agent, and a composition comprising one or more therapeutic agents that perturb the interaction between integrin β4 (ITGB4) and paxillin (PXN), e.g., by inhibiting the expression of ITGB4, PXN, or both. In some embodiments, the one or more therapeutic agents include an anti-ITGB4 antibody, an siRNA that inhibits ITGB4 expression, an anti-PXN antibody, and/or an siRNA that inhibits PXN expression. In some embodiments, the chemotherapeutic agent includes cisplatin and carboplatin. In some embodiments, the chemotherapeutic agent is administered at a reduced dose when administered in combination with the composition comprising the one or more therapeutic agents that perturb the interaction between integrin β4 (ITGB4) and paxillin (PXN) comparing to the dose of the chemotherapeutic agent administered alone. In some embodiments, administering the combination of the chemotherapeutic agent and the therapeutic agents that perturb the interaction between integrin β4 (ITGB4) and paxillin (PXN) achieves a synergistic effect. In some embodiments, the composition comprises one or more compounds selected from carfilzomib, ixazomib, and CUDC-101. In some embodiments, the subject suffers from one or more solid tumors. In some embodiments, the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma. In some embodiments, the subject has developed or is at an elevated risk of developing resistance to platinum-based chemotherapy. In some embodiments, the subject has developed or is at an elevated risk of developing resistance to cisplatin or carboplatin.

In another aspect, disclosed herein is a method of detecting resistance to chemotherapy in a subject who is subjected to chemotherapy. The method entails measuring the expression level of ITGB4, PXN, or both in a sample obtained from the subject, and comparing the expression level of ITGB4, PXN, or both with the expression level of ITGB4, PXN, or both in a sample obtained from a healthy control, wherein an elevated expression level of ITGB4, PXN, or both comparing to the healthy control indicating resistance to chemotherapy in the subject. Alternatively, the method entails measuring the expression level of ITGB4, PXN, or both in a sample obtained from the subject before and after the subject is subjected to chemotherapy, and comparing the expression level of ITGB4, PXN, or both after chemotherapy with the expression level of ITGB4, PXN, or both before chemotherapy, wherein an elevated expression level of ITGB4, PXN, or both after chemotherapy indicating resistance to chemotherapy in the subject. In some embodiments, the sample is serum. In some embodiments, the sample is exosomes isolated from serum. In some embodiments, the expression level is measured by Western Blot. In some embodiments, the subject suffers from one or more solid tumors. In some embodiments, the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma. In some embodiments, the subject has developed or is at an elevated risk of developing resistance to platinum-based chemotherapy. In some embodiments, the subject has developed or is at an elevated risk of developing resistance to cisplatin or carboplatin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F show that PXN and ITGB4 are upregulated in lung cancer and in cisplatin-resistant LUAD cell lines. FIG. 1A: Five LUAD cell lines with wild type (WT) KRAS and 5 cell lines with mutant (MT) KRAS were treated with 10 μM cisplatin for 72 hours and cell viability was determined using Cell Counting Kit-8 assay. FIG. 1B: Immunoblot showing that the most cisplatin-resistant cell lines (H1993 and H2009) had high expression of both PXN and ITGB4. FIGS. 1C and 1D: qPCR results demonstrating high mRNA expression of PXN and ITGB4 in cisplatin-resistant cell lines. FIG. 1E: Gene expression profiles of LUAD patients were extracted from TCGA database and expression of PXN and ITGB4 were higher compared to normal tissue. FIG. 1F: Kaplan-Meier curves indicate significant survival difference (p=0.000089) between the two groups (PXN low+ITGB4 low and PXN high+ITGB4 high).

FIGS. 2A-2B show variability of expression of PXN and ITGB4 in tumor tissue samples. FIG. 2A: Immunohistochemistry staining of needle biopsy specimens obtained from 2 de-identified cases from City of Hope. PXN staining was detected using yellow and ITGB4 with pink color. Thus, the coexpression of the two molecules would generate an orange or bright red color depending on the expression level. FIG. 2B: IHC staining to 3 more cases where staining of the whole tumor section was done. Case 3 PXN expression was intermediate and ITGB4 expression was high. Case4 had both intermediate expression for PXN and ITGB4 and in Case 5, both PXN and ITGB4 expression were low.

FIGS. 3A-3L show that knocking down ITGB4 attenuates proliferation and migration. FIG. 3A: Stable cell lines were generated to express nuclear mKate2 red fluorescent protein (RFP) using the NucLight Red Lentivirus Reagent (Essen BioScience). Upon selection with puromycin, cells were analyzed with the IncuCyte software to create a mask around each individual nucleus and obtain accurate real-time cell counts for proliferation assays. FIG. 3B: ITGB4 knockdown cells (red) had a significantly reduced proliferation rather than control cells (black). (**** p<0.0001 two-way ANOVA). FIG. 3C: Doubling times for H2009 and H1993 cells transfected with scramble siRNA (siControl) were measured and compared to that of cells with ITGB4 knockdown (siITGB4). FIG. 3D: Immunoblotting and qPCR data confirming the knockdown. FIG. 3E: Scratch wound healing assays were performed by creating an initial scratch wound with the WoundMaker tool. Wound closure was quantitated by monitoring cells that migrated to fill the initial wound. FIG. 3F: A scratch wound assay demonstrating the effect of knocking down ITGB4. ITGB4 knockdown (red) significantly halted migration and did not close the wound completely after 96 hours in both resistant cell lines. (**** p<0.0001 two-way ANOVA). FIG. 3G: Rate at which the wound closed was also significantly decreased in ITGB4 knockdown cells (red). (**** p<0.0001 and * p<0.0156 two-way ANOVA). FIG. 3H: H2009 cells transfected with 10 nM of siRNA constructs A, B, and C, targeting ITGA7 had minimal effect on proliferation. FIG. 3I: Knocking down ITGB4 increased mRNA expression of other integrin beta forms such as ITGB1, ITGB2, and ITGB3 but had no significant effect on the expression of ITGA7. FIG. 3J: To nullify the effect of ITGB3 rescue, H2009 cells transfected with siRNA ITGB4 were treated with ITGB3 inhibitors. There was no significant effect in the fold change in proliferation compared to the ITGB4 knockdown cells. FIGS. 3K and 3L: ITGB4 knockdown alone induces varying effects in 1650 cell lines. Nuclear RFP-expressing cell line H1650 was transfected with ITGB4 siRNA (si ITGB4) and monitored in real-time with the IncuCyte. Over the course of 10 hours, ITGB4 knockdown induced cells with an intact membrane to undergo anoikis-like bursting, attenuating cell proliferation in Si ITGB4 (red) cells compared to control (black).

FIGS. 4A-4I show that knocking down ITGB4 increases sensitivity of cisplatin-resistant cells. FIG. 4A: Cisplatin (10 μM) treatment for 72 hours reduced expression in phosphorylated and total FAK and PXN but not ITGB4. FIG. 4B: Proliferation and apoptosis assays were executed with stable cell lines expressing nuclear RFP and the IncuCyte Caspase-3/7 Green Reagent (Essen BioScience), which emits green fluorescence when cleaved by activated caspase-3/7. Apoptosis was induced with knockdown of ITGB4 (Si ITGB4) by day 4 and enhanced with added cisplatin (Si ITGB4+Cisplatin). FIGS. 4C and 4D: ITGB4 knockdown in H1993 cells inhibited proliferation and increased caspase-3/7 activity. Treating ITGB4 knockdown cells with cisplatin had an additive effect to induce caspase activity but drug treatment alone did not have a cytotoxic effect. (****p<0.0001 Two-way ANOVA) FIGS. 4E and 4F: ITGB4 knockdown in H2009 cells inhibited proliferation by 72 hours but did not induce caspase activity. Cisplatin treatment to ITGB4 knockdown cells for 72 hours had an additive effect to induce caspase activity. (****p<0.0001 Two-way ANOVA) FIG. 4G: Immunoblot showing MET protein expression in H1993 cells was reduced 4 days after knocking down ITGB4. FIG. 4H: H1993 cells harboring MET amplification and upon ITGB4 knockdown, MET expression at only the protein level decreased but no change in the mRNA expression, indicating ITGB4 is required for MET protein stability but not for transcriptional regulation. Thus, MET downregulation upon ITGB4 knockdown possibly increased apoptosis significantly in MET signaling dependent cells compared to control cells. FIG. 4I: H2009 cells that did not have amplified MET did not show any significant change in the protein as well as mRNA expression. They also did not show significant increase in apoptosis with ITGB4 knockdown.

FIGS. 5A-5M show that neutralizing ITGB4 with an antibody or knocking down both PXN and ITGB4 has a synergistic effect on attenuating cisplatin resistance. FIGS. 5A and 5B: H1993 cells were treated with an antibody (10 μg/ml) targeting the ITGB4 extracellular epitope in an ultra-low attachment 96-well plate for 6 hours then transferred to a tissue culture-treated 96-well plate and allowed to attach overnight. After 96 hours, proliferation was inhibited and apoptosis was significantly induced in cells treated with the ITGB4 antibody (red) compared to untreated (black) and cells treated with an IgG antibody (green) used as a control. (****p<0.0001 Two-way ANOVA, multiple comparison) FIG. 5C: Immunoblotting confirmed decreased expression of ITGB4, PXN, and MET with ITGB4 antibody treatment after 48 hours compared to IgG antibody control. FIGS. 5D and 5E: ITGB4 antibody (10 μg/ml) treatment in combination with a lower dose of cisplatin (2.5 μM instead of 10 μM) exhibited a synergistic effect on inhibition of proliferation and increased caspase activity. (****p<0.0001 Two-way ANOVA) FIG. 5F: Double knockdown of both PXN and ITGB4 (red) in H1993 cells had a synergistic effect on inhibiting proliferation compared to single knockdown of either gene (green/blue) and control (black). FIG. 5G: Adding 10 μM cisplatin to the double knockdown cells (red) had an even greater effect on inhibiting proliferation compared to single knockdown of either gene (green/blue) and control (black) treated with cisplatin. (****p<0.0001 Two-way ANOVA) FIG. 5H: ITGB4 knockdown and double knockdown of PXN and ITGB4 induced apoptosis in H1993 and rendered cells more prone to toxic effects of cisplatin. (****p<0.0001 Two-way ANOVA) FIG. 5I: Double knockdown of both PXN and ITGB4 (red) in H2009 cells also had a synergistic effect on inhibition of proliferation compared to single knockdown of either gene (green/blue) and control (black). (****p<0.0001 Two-way ANOVA) FIGS. 5J and 5K: Double knockdown of PXN and ITGB4 also induced strong apoptosis and in combination with cisplatin, proliferation was greatly inhibited and caspase activity increased at an earlier time point of 24 hours. (****p<0.0001 Two-way ANOVA) FIG. 5L: Immunoblotting confirmed siRNA-mediated knockdown of PXN and ITGB4. PXN knockdown alone increased expression of p27 and decreased levels of phospho-Rb (S807/811), indicating cell cycle arrest. FIG. 5M: Cell cycle analysis revealed that knocking down PXN induced G1-S arrest whereas knocking down ITGB4 arrested cells in G2-M. Double knockdown of PXN and ITGB4 arrested cells in G1-S and G2-M.

FIGS. 6A-6J show that both PXN and ITGB4 are necessary for spheroid viability and their signaling pathways converge in cisplatin resistance. H2009 cells expressing RFP were transfected with siRNA, seeded in a 96-well ultra-low attachment plate (5000 cells/well), and allowed to form a compact spheroid overnight. FIG. 6A: H2009 cells expressing nuclear RFP were seeded in an ultra-low attachment 96-well plate to facilitate spheroid formation. After 4 hours, double knockdown of PXN and ITGB4 impeded cells from forming a compact spheroid as observed in control and single knockdown conditions. FIG. 6B: Images acquired by the IncuCyte Live Cell Imaging System showed spheroids with single knockdown to start disintegrating by Day 3 and even earlier for double knockdown spheroids (Day 1). FIG. 6C: Immunoblotting confirmed that PXN and ITGB4 siRNA-mediated knockdown was still present after 72 hours in 3D culture. FIGS. 6D and 6E: To quantitate spheroid viability, red fluorescence area and intensity were measured. Both parameters showed that double knockdown had a synergistic effect on attenuating spheroid viability. (****p<0.0001 Two-way ANOVA) FIGS. 6F and 6G: Spheroids with ITGB4 single knockdown and PXN/ITGB4 double knockdown were sensitized to cisplatin (10 μM) treatment indicated by a decrease in red fluorescence area and intensity. (****p<0.0001 Two-way ANOVA) FIG. 6H: Double knockdown spheroids treated with cisplatin had the greatest cytotoxic effect indicated by the green fluorescence in the confocal images acquired by a Zeiss LSM 880 microscope. (**p<0.002, ***p<0.0009 Two-way ANOVA) FIG. 6I: In H2009 cells, single knockdown of ITGB4 (Si ITGB4) significantly induced apoptosis and double knockdown of PXN and ITGB4 (Si ITGB4+Si PXN) had an enhanced effect. FIG. 6J: Total RNA was extracted from single and double knockdown cells 48 hours after siRNA transfection. Total RNASeq revealed the number of genes downregulated with each single knockdown of PXN and ITGB4 and when both genes are knocked down simultaneously. FIG. 6K: Bar diagram showing the major Hallmark pathways affected by the double knockdown. The top 10 pathways were arranged in descending order of their enrichment score. FIG. 6L: Heat map representation of top 10 genes that belong to hallmark MYC target V1 pathways were analyzed after single or double knockdown. The heat map is representation of two experimental repeats.

FIGS. 7A-7G show the genes and the signaling pathways inhibited by both ITGB4 and PXN knockdown. FIG. 7A: Total RNA sequencing after single and double knockdown of PXN and ITGB4 revealed that 5 common genes were downregulated compared to control cells (SCR). FIG. 7B: Major pathways affected by double knockdown of PXN and ITGB4 were MYC targets, G2-M checkpoint, and E2-F targets. FIG. 7C: qPCR results with single and double knockdown of PXN and ITGB4 confirmed downregulation of VDAC1, USP1, and G3BP1 at the mRNA level. FIG. 7D: SiRNA constructs A, B, or C against top three downregulated genes (VDAC1, USP1, and G3BP1) were tested to determine the construct with maximum effect on inhibiting proliferation. VDAC1 construct A and USP1 construct B showed significant effect on cell proliferation. FIG. 7E: The selected constructs were tested for induction of caspase-3/7 activity by tracking green fluorescence using live cell imaging and analysis for 72 hours. FIG. 7F: Immunoblotting confirmed decreased expression of top 3 genes (G3BP1, USP1, and VDAC1) downregulated in MYC pathway after knockdown of PXN and/or ITGB4. FIG. 7G: Gene expression profiles of LUAD patients were extracted from TCGA database and expression of USP1 and VDAC1 were higher compared to normal tissue.

FIGS. 8A-8O show that both PXN and ITGB4 are regulate expression of USP1 and VDAC1 genes which are required for maintaining genomic stability and mitochondrial function. FIGS. 8A and 8B: In H2009 cells, knocking down USP1 (blue) attenuated proliferation, induced apoptosis, and sensitized cells to a lower dose of cisplatin (2 μM) (red). (****p<0.0001 Two-way ANOVA) FIG. 8C: After knocking down PXN/ITGB4 and USP1, γH2AX foci were detected via immunofluorescence, imaged with confocal microscopy, and counted with QuPath image analysis software. Knockdown exhibited greater number of cells with higher γH2AX foci counts. Added cisplatin increased number of detected γH2AX foci. (*p=0.04, **p=0.004, ***p,<0.0001) FIG. 8D: H2009 and H1993 cells treated with ML323, a USP1 inhibitor, did not undergo significant changes in proliferation compared to untreated cells (black), indicating blocking the interaction of USP1 with its partners did not induce apoptosis rather the reduction in USP1 protein level enhanced sensitivity. FIGS. 8E and 8F: Similarly, knocking down VDAC1 (blue) inhibited cell proliferation by 50% within 72 hours, but could induce apoptosis only at later time point. Cisplatin (red) addition had an additive effect to VDAC1 knockdown in inhibiting proliferation. FIGS. 8G-8K: Using the Seahorse XF Analyzer, cellular metabolic activity was measured with knockdown and in the presence of cisplatin. Knocking down PXN/ITGB4 or VDAC1 increased basal (8H) and maximal (8I) mitochondrial oxygen consumption rate per 1000 cells. (**p=0.0029, ****p<0.0001 One-way ANOVA). ATP-linked respiration (8J) and proton leak (8K) also showed an increase in the same knockdown cells. (*p=0.01 One-way ANOVA). FIG. 8L: Double knockdown of PXN/ITGB4 (red) and USP1/VDAC1 (blue) inhibited proliferation to similar extent whether in the absence or presence of cisplatin. (****p<0.0001 Two-way ANOVA) FIG. 8M: Immunoblot shows no changes in the expression of ITGB4 and PXN expression after knocking down USP1 and VDAC1 in H2009 and H1993 cell lines. FIG. 8N: Reactive oxygen species (ROS) production was measured using the ROS-Glo™ H₂O₂ Assay. Double knockdown of PXN/ITGB4 and USP1/VDAC1 induced higher levels of ROS compared to control. FIG. 8O: ChIP was performed with an acetylated H3K27 antibody 72 hours after siRNA-mediated knockdown of PXN/ITGB4. With the knockdown, H3K27 acetylation at the promoter region of USP1 was greatly reduced compared to that of an upstream region, indicating ITGB4 and PXN role in regulating USP1 transcriptional activation.

FIGS. 9A-9L show that PXN is intrinsically disordered and its interaction with ITGB4 can be disrupted by an FDA-approved compound Carfilzomib. FIG. 9A: Co-immunoprecipitation (co-IP) of H2009 whole cell lysate with an ITGB4 antibody showing FAK, PXN, and ITGA7 interaction with ITGB4. Reverse co-IP with FAK and PXN antibody showing ITGB4 binding to FAK and PXN. FIG. 9B: Knocking down PXN did not affect the interaction between FAK and PXN. FIGS. 9C and 9D: Treating H2009 cells with 10 μM cisplatin for 48 hours did not induce any changes in the interaction between ITGB4, FAK, and PXN. FIG. 9E: PONDR prediction algorithm determined that the N-terminal region of PXN to be intrinsically disordered whereas the C-terminal half is highly ordered. FIG. 9F: Circular dichroism (CD) spectra were recorded as a function of temperature 10° C. to 60° C. Low ellipticity values near the 215-230 nm region indicated that the N-terminal region lacks any secondary structure. FIG. 9G: NMR spectroscopy using a two-dimensional 1H-15N HSQC spectrum was used to analyze the LD2-LD4 region of PXN to confirm that it is indeed disordered. FIG. 9H: Using the binding pocket in the N-terminal region of PXN, 1440 FDA-approved drugs were virtually screened. FIG. 9I: Eleven FDA-approved compounds that exhibited the greatest inhibitory effect on H358 cells were further analyzed with immunoblot. H358 cells were treated with each compound at the indicated sublethal dose for 72 hours. FIGS. 9J and 9K: H2009 cells treated with the eleven FDA-approved compounds at 0.08 μM. Cell proliferation over the course of 72 hours was monitored and viability percentage compared to untreated was measured using CCK-8 assay. Carfilzomib showed highest toxicity with a 80% reduction in the cell viability. FIG. 9L: Molecular structure of carfilzomib.

FIGS. 10A-G: FIG. 10A: H2009 cells were treated with an increasing dose of carfilzomib for 72 hours to determine IC50, which is identified to be 0.418 uM. FIG. 10B: Scratch wound assay using H2009 cells treated with a sublethal dose of carfilzomib (50-200 nM) showed dose-dependent inhibition of cell migration. (**p-0.002, ***p-0.0001, p-****<0.0001) At a dose of 200 nM, the wound is 79% confluent compared to >90% confluency for untreated cells. FIG. 10C: Immunoblot of H2009 cells treated with carfilzomib showed reduced expression of ITGB4 and phospho-Rb (S807/811) and increased expression of p27, γH2AX, and cleaved PARP, indicating cell cycle inhibition and cell death. FIG. 10D: H1993 cells were treated with an increasing dose of carfilzomib to determine cell proliferation effect with change in dose and determine the IC50, which was identified to be 1.77 uM. FIG. 10E: Immunoblot of H1993 cells treated with carfilzomib showed reduced expression of ITGB4 and phospho-Rb (S807/811) and increased expression of p27, γH2AX, and cleaved PARP, indicating cell cycle inhibition and cell death. FIGS. 10F and 10G: Ixazomib and CUDC-101, two other potent compounds that induced caspase activity, were further analyzed with scratch wound healing assays in H2009 cells by taking into parameters relative wound density and wound closure rate. Only Ixazomib impeded cell migration and CUDC-101 did not.

FIGS. 11A-11J show that carfilzomib is more effective than cisplatin in 3D spheroids and patient-derived organoids. FIG. 11A: Confocal images taken of H2009 spheroids treated with 300 nM and 600 nM carfilzomib for 72 hours depicted disintegration and induction of caspase activity marked by green fluorescence. FIGS. 11B and 11C: Carfilzomib treatment of H2009 spheroids reduced viability of spheroids as quantitated by lower red fluorescence intensity (11B) and increased caspase activity (11C) in a dose-dependent manner. At a dose of 300 nM the red intensity of spheroid dropped by 2-fold and caspase activity increased by 2 fold. (****p<0.0001 Ordinary one-way ANOVA) FIGS. 11D-11J: Surgical samples from lung cancer patients were obtained and cultured to form organoids (5000 cells/well). FIG. 11D: Image of patient (LCSC4) derived spheroids after treatment with carfilzomib or cisplatin or both. FIG. 11 E: Cisplatin (5 μM) alone or carfilzomib (2.5 μM) alone decreased the size of the organoid. FIG. 11F: 5-fold greater induction of apoptosis with carfilzomib compared to cisplatin in reference to cisplatin, (****p<0.0001 Two-way ANOVA) The combination of carfilzomib and cisplatin did not have an additive effect. FIG. 11G: Image of patient (LCSC2) derived spheroids after treatment with carfilzomib or cisplatin or both. FIG. 11H: cisplatin and carfilzomib decreased the spheroid area. FIG. 11I: Cisplatin alone did not induce any caspase activity but carfilzomib induced a ˜50-fold increase in apoptosis. (****p<0.0001 Two-way ANOVA). Caspase activity was enhanced with the combination of both drugs. (****p<0.0001 Two-way ANOVA) FIG. 11J: These patient-derived organoids were treated with cisplatin and carfizomib at indicated concentrations and collected after 48 hours for immunoblot. Cisplatin treatment had a minimal effect whereas carfilzomib had a more toxic effect on cells marked by decreased expression of ITGB4, PXN, USP1, VDAC1 and CD133.

FIGS. 12A-12F are mathematical modeling suggesting that cisplatin resistance can be stochastic and reversible state, and ITGB4 and PXN detected in exosomes represent attractive biomarkers. FIG. 12A: A double negative feedback loop between ITGB4 and miR-1-3p leads to bistability. FIG. 12B: A mathematical model stimulating the dynamics of ITGB4 and miR-1-3p showed cisplatin resistance to be a reversible state. High ITGB4 and low miR-1-3p render cells to be resistant whereas low ITGB4 and high miR-1-3p represents a more sensitive state. FIG. 12C: To test this mathematical model, RACIPE algorithm generated an ensemble (n=100,000) with varying parameter sets then plotted to represent robust dynamical patterns. Results showed that ITGB4 and miR-1-3p exhibited bimodality: two distinct subpopulations of cells that were negatively correlated, reinforcing the previously described negative feedback loop. FIG. 12D: Human sera (500 μl) were obtained from healthy donors and LUAD patients. Exosomes were isolated, lysed, and analyzed for expression of ITGB4 and PXN by immunoblotting. Compared to that of healthy donors, exosomes of patients had increased expression of ITGB4 and PXN and also USP1, which were identified to be regulated by ITGB4. FIG. 12E: H2009 cells were stained with ITGB4 antibody conjugated to Alexa Fluor 488 and sorted based on gates set to high and low ˜10% of ITGB4-expressing population using the FACSAria Fusion instrument. Sorted cells were subsequently cultured for 48 hours and then treated with 1 μM cisplatin for 48 hours. Then using the Attune NxT Flow Cytometer, equal numbers of cells were stained again and analyzed to determine shifts in population between untreated and treated cells. Low sorted cells treated with cisplatin had a greater cell population that shifted toward higher ITGB4 expression. High sorted cells treated with cisplatin did not undergo significant changes in population compared to untreated. FIG. 12F: Schematic depicting the interaction between ITGB4 and PXN regulating downstream proteins USP1 and VDAC1 at the transcriptional level to coordinate cisplatin resistance.

FIGS. 13A-13B show FACS analysis using an anti-ITGB4 antibody showed enrichment of high ITGB4 population upon cisplatin treatment in H2009 cells.

### DETAILED DESCRIPTION

Disclosed herein is a method of alleviating resistance to chemotherapy in a subject, the method includes administering to the subject a composition comprising one or more therapeutic agents that perturb the interaction between ITGB4 and PXN, e.g., by inhibiting the expression of ITGB4, PXN, or both. Also disclosed is a combinational therapy, comprising co-administration of a chemotherapy, e.g., a platinum-based chemotherapy such as cisplatin or carboplatin, and one or more therapeutic agents that perturb the interaction between ITGB4 and PXN such as carfilzomib, ixazomib, and CUDC-101. As used herein, “co-administration” means that the chemotherapeutic agent and the composition are administered simultaneously or within a short interval (e.g., within a few hours, a few days, or a few weeks) before or after the subject has developed or shows sign of resistance to chemotherapy. Moreover, the composition can be administered before or after administration of the chemotherapeutic agent.

This disclosure identifies a new therapeutic target and FDA-approved drugs that can potentially be repurposed to alleviate cisplatin resistance together with a minimally invasive biomarker assay, can have a significant impact on LUAD. These findings also underscore an alternate, non-genetic mechanism underlying the evolution of chemoresistance, which can alter the treatment of advanced-stage lung cancers that present with limited therapeutic options.

In lung adenocarcinoma (LUAD), PXN is associated with cisplatin resistance. As demonstrated herein, a significant region of the N-terminal half of PXN is intrinsically disordered and interacts with integrin beta 4 (ITGB4). Silencing PXN or ITGB4 augments cisplatin sensitivity and silencing both molecules has a synergistic effect. Immunologically neutralizing ITGB4 activity also improves cisplatin resistance. By screening an FDA-approved compound library, identified are compounds that interact with PXN in silico and attenuate cisplatin resistance in LUAD cells. RNAseq analysis identified a double negative feedback loop between ITGB4 and microRNA miR-1-3p, suggesting that bistability can lead to stochastic switching between cisplatin-sensitive and resistant states in these cells. The data highlight an alternate, non-genetic, mechanism underlying chemoresistance in lung cancer.

The role of the focal adhesion (FA) complex in cisplatin resistance was investigated in this study. The FA complex is a large macromolecular assembly through which mechanical force and regulatory signals are transmitted between the extracellular matrix and an interacting cell (Chen et al, 2003). Paxillin (PXN), integrins, and focal adhesion kinase (FAK) are among the major components of this complex. Human PXN is a 68 kDa (591 amino acids) protein (Salgia et al, 1995) and is a recognized contributor to cisplatin resistance in lung cancer (Wu et al, 2014). The N-terminus contains a proline-rich region that anchors SH3-containing proteins and five leucine-rich LD domains (LD1-LD5) with a consensus sequence LDXLLXXL (SEQ ID NO:1) (Turner, 1998; Turner, 2000; Kanteti et al, 2016). The LD2-LD4 region includes sequences for the recruitment of signaling and structural molecules, such as FAK, vinculin, and Crk. This region has also been reported to interact with integrin α, more specifically, integrin α4 (ITGA4) (Liu et al, 1999; Liu and Ginsburg, 2000). The C-terminal region is also involved in the anchoring of PXN to the plasma membrane and its targeting to FAs. It contains four cysteine-histidine-enriched LIM domains that form zinc fingers, suggesting that PXN could bind DNA and act as a transcription factor. Consistently, PXN is reported to locate to the nucleus which is regulated by phosphorylation (Dong et al, 2009; Ma and Hammes, 2018). In LUAD, expression of PXN is correlated with tumor progression and metastasis (Song et al, 2010; Mackinnon et al, 2011). Further, phosphorylation of PXN activates the ERK pathway, increased Bcl-2 expression, and cisplatin resistance (Wu et al, 2014). Finally, specific PXN mutants, through their interactions with Bcl-2 and dynamin-related protein 1, also regulate cisplatin resistance in human lung cancer cells (Kawada et al, 2013).

Integrins are transmembrane receptors that facilitate cell-extracellular matrix adhesion; they form a critical link between the extracellular matrix and the cell interior by interacting with the FA via PXN. Upon ligand binding, integrins activate signal transduction pathways that mediate cellular signals, such as regulation of the cell cycle, organization of the intracellular cytoskeleton, and movement of new receptors to the cell membrane (Giancotti and Rusolahti, 1999; Maziveyi and Alahari, 2107). Integrins are obligate heterodimers of one α and one β subunit. In mammals, there are 24 α and 9 β subunits (Alberts et al, 2014). Among the various β subunits, β1 is ubiquitously expressed in most cell types and can dimerize with multiple a subunits, forming receptors for various matrixes. On the other hand, integrin β4 (ITGB4) is reported to be quite selective and heterodimerizes only with the α6 subunit and binds to laminin (Mainiero et al, 1997). ITGB4 is also unique because of its >1000 amino acid-long cytoplasmic domain compared to ˜50 amino acid-long domain of other β forms (Su et al, 2008). Interaction of ITGA6/B4 and Shc leads to activation of the RAS-MAPK signaling pathway for cell cycle progression and proliferation (Mainiero et al, 1995). ITGA6/B4 can also activate the PI3 kinase pathway followed by Rac1 to promote tumor invasion (Shaw et al, 1997). In NSCLC, the receptor tyrosine kinase MET interacts with ITGA6/B4 and this interaction is required for HGF-dependent tumor invasion (Trusolino et al, 2001). In addition, the presence of the ITGA6/B4 heterodimer in tumor-derived exosomes facilitates the creation of the microenvironment for lung metastasis (Hoshino et al, 2015). Together, these observations underscore the importance of the FA complex in NSCLC pathophysiology. However, how the interactions of the individual components of the FA complex may contribute to cisplatin resistance remains poorly understood.

It is generally held that the antineoplastic effects of cisplatin are due to its ability to generate unrepairable DNA lesions hence inducing either a permanent proliferative arrest (cellular senescence) or cell death due to apoptosis. The drug enters cells via multiple pathways and forms multiple DNA-platinum adducts which results in dramatic epigenetic and/or genetic alternations. Such changes have been reported to occur in almost every mechanism supporting cell survival, including cell growth-promoting pathways, apoptosis, developmental pathways, DNA damage repair, and endocytosis (Shen et al, 2012; Rocha et al, 2018). Therefore, at a single cell level, the genetic underpinning involved in cisplatin resistance is obvious; however, the exact molecular mechanism(s) underlying the emergence of cisplatin resistance remains poorly understood and a bewildering plethora of targets have been implicated in different cancer types.

On the other hand, drug resistance is also thought to be strongly influenced by intratumoral heterogeneity and changes in the microenvironment (Alvarez-Arenas et al, 2019). Again, while prevailing wisdom advocates that the heterogeneity arises from genetic mutations, and analogous to Darwinian evolution, selection of tumor cells results from the adaptation to the microenvironment (Gerlinger M, Swanton, 2010), it is now increasingly evident that non-genetic mechanisms may also play an important role and information transfer can occur horizontally via a Lamarckian mode of evolution (Alvarez-Arenas et al, 2019). Thus, a population of isogenic cells in the same environment can exhibit single-cell-level stochastic fluctuations in gene expression. Such fluctuations, known as gene expression noise or transcriptional noise, can result in isogenic cells ‘making’ entirely different decisions with regard to their phenotype and hence, their ability to adapt themselves to the same environmental perturbation (Balázsi et al, 2011; Farquhar et al, 2019; Engl, 2019). Transcriptional noise can arise from the intrinsic randomness of underlying biochemical reactions or processes extrinsic to the gene (Swain et al, 2002). Regardless, two main characteristics of gene expression noise are its amplitude and memory. Amplitude, often measured by the coefficient of variation, defines how far cells deviate from the average. Memory describes the time for which cells remain deviant once they depart from the average (Acar et al, 2005; Charlebois et al, 2011). Thus, it follows that the effect noise produces is likely reversible and hence, underscores its importance in phenotypic switching.

Yet another source of noise that can be confounding in phenotypic switching is conformational noise (Mahmoudabadi et al, 2013) that stems from the ‘structural’ plasticity of the IDPs that lack rigid 3D structure and exist as conformational ensembles instead (Wright and Dyson, 2015; Turoverov et al, 2019). Because of their conformational dynamics and flexibility, IDPs can interact with multiple partners and are typically located in ‘hub’ positions in protein interaction networks. The collective effect of conformational noise is an ensemble of protein interaction network configurations, from which the most suitable can be explored in response to perturbations. Moreover, the ubiquitous presence of IDPs as transcriptional factors (Staby et al, 2017; Tsafou et al, 2018), and more generally as hubs (Patil et al, 2010; Hu et al, 2017), underscores their role in propagation of transcriptional noise. As effectors of transcriptional and conformational noise, IDPs rewire protein interaction networks and unmask latent interactions (Mahmoudabadi et al, 2013). Thus, noise-driven activation of latent pathways appears to underlie phenotypic switching events such as drug resistance.

As demonstrated herein, a significant portion of the N-terminal half of PXN is intrinsically disordered and that the interaction between ITGB4 and PXN is critical for cisplatin resistance. ITGB4 and PXN double knockdown affects the expression of >300 genes which are constituents of various pathways required for lung cancer proliferation and survival. USP1 and VDAC1 are two of the top ten genes that are downregulated by the double knockdown that are essential for inducing DNA damage repair induced by cisplatin and for maintaining the mitochondrial function, respectively. Via an in silico screen designed to identify small molecule compounds that can bind to PXN, candidate drugs were obtained from a library of FDA-approved compounds. Several of these compounds are efficacious in alleviating cisplatin resistance in LUAD cell lines expressing high levels of ITGB4/PXN. Furthermore, using tumor-derived exosomes isolated from patient blood, ITGB4/PXN were identified as potential biomarkers for cisplatin response. Moreover, RNAseq analysis identified a double negative feedback loop between ITGB4 and the microRNA miR-1-3p, suggesting that bistability can lead LUAD cells to stochastically switch between cisplatin-resistant and sensitive phenotypes, underscoring a non-genetic mechanism driving the evolution of chemoresistance.

The present data imply that cisplatin resistance in LUAD can arise stochastically in response to drug treatment. While it is possible that such resistance that is spontaneously, or randomly, acquired during the course of treatment can be due to random genetic mutations or stochastic non-genetic phenotype switching (Pisco et al, 2013), these observations strongly support a non-genetic mechanism. A double negative feedback loop between ITGB4 and miR-1-3p results in bistability, facilitating a reversible phenotypic switch between cisplatin-sensitive and resistant states. A recent study on oxaliplatin chemotherapy in pancreatic ductal adenocarcinoma (Kumar et al, 2019) where a coarse-grained stochastic model to quantify phenotypic heterogeneity in a population of cancer cells was studied. The present findings suggest that the phenomenon may be applicable to many different cancers. Furthermore, a random population of cisplatin-resistant LUAD cells is heterogeneous and comprises individuals that either express high or low levels of ITGB4. These cells therefore are either more resistant or less resistant to cisplatin, respectively. However, when purified to homogeneity (>99%) and plated separately, the purified population recreates the heterogeneity. Taken together, these observations not only corroborate the bistability predicted by the model but also highlight the role of phenotypic switching in generating population heterogeneity in cancer.

Additionally, the interaction of ITGB4 with the intrinsically disordered PXN is critical since perturbing this interaction renders the cells sensitive. It is important to note that, in addition to the LD domains and the LIM domains, PXN also contains an SH3 domain-binding site and SH2 domain-binding sites (Salgia et al, 1995). Together, these motifs serve as docking sites for cytoskeletal proteins, tyrosine kinases, serine/threonine kinases, GTPase activating proteins, and a host of other adaptor proteins that recruit additional enzymes into complex with PXN. Thus, consistent with the functions of an IDP in a hub position, PXN serves as a docking protein to recruit signaling molecules to the FA complex and thereby, coordinate downstream signaling (Schaller, 2001, Oncogene). It is now well recognized that cellular protein interaction networks are organized as scale-free networks and hence, are remarkably resilient to perturbations (Barabasi and Albert, 1999; Barabasi, 2009). Thus, while disabling minor nodes does not significantly affect the continuity and hence, functionality of the network, attacking the critical nodes can incapacitate the entire network (Schwartz et al, 2002). Thus, it follows that PXN appears to constitute a critical hub and its malfunction accounts for the failure of the cisplatin-resistant cells to tolerate the drug.

Although, IDPs in general have not been much appreciated as therapeutic targets since their inability to adopt well-defined structures provides significant obstacles for developing ligands that regulate their behaviors, emerging evidence indicates that indeed, they can be specifically targeted (Wojcik et al, 2018; Neira et al, 2017; Martin-Yken et al, 2016; Yu et al, 2016; Berg, 2011; Jung et al, 2015; Ambadipudi and Zweckstetter, 2016). In fact, even transcription factors that were never the favorite drug targets, are now emerging as tractable to drug development (Tsafou et al, 2018). Identified herein are a series of FDA-approved drugs including carfilzomib that perturb interactions involving an IDP and may be repurposed for alleviating cisplatin resistance in LUAD. By extrapolation, it is likely that some of these drugs may be effective in several other cancer types in which cisplatin therapy is administered. Thus, carfilzomib and the other drugs identified in this study can not only hasten clinical trials in future, but may make the availability of the drug more cost effective as well. Additionally, using the antibody-drug conjugation technology, carfilzomib or any of the other drugs identified in this study can be conjugated to the ITGB4 antibody and delivered to the tumor site with high specificity (Yao et al, 2016).

The role of the FA complex in serving as a conduit through which mechanical force and regulatory signals are transmitted between the extracellular matrix and an interacting cell is well established (Chen et al, 2003). Furthermore, the role of PXN in cisplatin resistance has also been recognized (Wu et al, 2014). However, the involvement of ITGB4 and the interaction between these molecules with FAK in modulating cisplatin resistance has not been reported. The findings disclosed herein support a new role for the FA complex in cancer, particularly LUAD. Thus, these FA components may serve as drug response predictors using a blood-based assay, and the identification of FDA-approved drugs that can be used to address drug resistance, which has a significant impact on LUAD and other types of cancers that respond to cisplatin.

The following examples are provided to better illustrate the claimed invention and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Materials and Methods

Cell lines and reagents: Lung cancer cell lines (H23, H358, SW1573, H441, H2009, H522, H1650, H596, H1437, and H1993) were obtained from American Type Culture Collection (ATCC) (Manassas, VA, USA). All cell lines were cultured in RPMI 1640 medium (Corning) supplemented with fetal bovine serum (FBS) (10%), L-glutamine (2 mM), penicillin/streptomycin (50 U/ml), sodium pyruvate (1 mM), and sodium bicarbonate (0.075%) at 37° C., 5% C02. Cisplatin was provided by City of Hope National Medical Center clinics. Anti-integrin beta 4 antibody, clone 8 was purchased from MilliporeSigma (Burlington, MA, USA). FDA-approved drugs were purchased from Selleck Chemicals (Houston, TX, USA).

Antibodies: Antibodies against ITGB4, FAK, phospho-FAK (Y397), γH2AX, p27, phospho-Rb (S807/811), USP1 were purchased from Cell Signaling Technology (Danvers, MA, USA). Antibodies against ITGA7, ITGA6, PXN, MET, G3BP1, VDAC1, and agarose-conjugated antibodies (ITGB4, FAK, PXN) were purchased from Santa Cruz Biotechnology (Dallas, TX, USA). Cyclin D1 antibody was purchased from Invitrogen (Waltham, MA, USA). Phospho-PXN (Y31) and PARP antibodies were purchased from Abcam (Cambridge, UK). CD63 antibody was purchased from System Biosciences (Palo Alto, CA, USA). β-actin antibody was purchased from Sigma-Aldrich (St. Louis, MO, USA).

West blotting: Cell lysates were prepared with 1×RIPA buffer (MilliporeSigma) and denatured in 1× reducing sample buffer at 95° C. for 5 minutes. Protein samples (15 μg) were run on 4-15% TGX gels (Bio-Rad, Hercules, CA, USA) and transferred onto nitrocellulose membranes (Bio-Rad). Blots were blocked with 5% non-fat milk in TBS-T for 1 hour at room temperature and probed with primary antibody diluted in 2.5% BSA in TBS-T overnight at 4° C. After three washes with TBS-T, blots were incubated with HRP-conjugated secondary antibodies for 2 hours at room temperature. After three more washes, bands of interest were visualized via chemiluminescence using Western Bright ECL HRP substrate (Advansta, Menlo Park, CA, USA) and imaged with the ChemiDoc MP imager (Bio-Rad).

Quantitative real-time PCR: Quantitative real-time PCR (qPCR) reactions were performed using TaqMan Universal PCR Master Mix (Thermo Fisher Scientific, Waltham, MA) and analyzed by the Quant Studio7 Real-time PCR system (Life Technologies, Grand Island, NY). Total RNA isolation and on-column DNase digestion from cells were performed basing on the manufacturer's protocol RNeasy Plus Mini Kit (Qiagen Cat #: 74134). 1 μg of RNA was used to synthesize the cDNA according to the one step cDNA synthesis kit from QuantaBio (Cat #: 101414-106). TaqMan probes for HS99999905-GAPDH, HS00236216-ITGB4, HS01104424-PXN, HS00174397-ITGB1, HS00164957-ITGB2, HS01001469-ITGB3, HS01565584-MET, HS04978484-VDAC1, HS00428478-G3BP1 and HS00163427-USP1 were purchased from ThermoFisher (Waltham, MA). The mRNA expression was analyzed using multiplex PCR for the targeted gene of interest and GAPDH as reference using two independent detection dyes FAM probes and VIC probes respectively. Relative mRNA expression was normalized to GAPDH signals and calculated using the ddCt method.

siRNA Transfection: Knockdown of ITGB4 (Cat #: SR302473C), FAK (Cat #: SR303877C), USP1 (Cat #: SR305052B), and VDAC1 (Cat #: SR305067C) at the mRNA level was executed using siRNAs purchased from OriGene Technologies (Rockville, MD, USA). Knockdown of PXN was achieved by siRNA purchased from Life Technologies Corporation (Cat #: 4392421). JetPRIME transfection reagent (Polyplus Transfection, Illkirch, France) was used to transfect the siRNAs according to the manufacturer's protocol. Cells were seeded in 6-well plates (200,000 cells/well) and allowed to adhere overnight. Next day, 10 nM siRNA was transfected with 4 μl jetPRIME reagent in complete growth medium for each well. Cell growth medium was changed the next day and expression was detected 72 hours post-transfection by immunoblot.

Cell viability assay: Cell Counting Kit-8 (CCK-8) was purchased from Dojindo Molecular Technologies (Rockville, MD, USA). Cells were seeded on a 96-well plate and allowed to adhere in complete medium for 24 hours. Test compounds were added to 100 μl of medium at the indicated concentrations for 72 hours. Ten μl of the CCK-8 reagent were added to each well and absorbance at 450 nm was measured using a Tecan Spark 10M multimode microplate reader.

Scratch wound healing assay: Cells were seeded on a 96-well Image Lock (Essen BioScience, Ann Arbor, MI, USA) plate to reach 90% confluence by the next day. After cell adherence, 96 uniform wounds were created simultaneously using the WoundMaker (Essen BioScience) tool. Cells were washed once with serum-free medium and replenished with complete medium. To monitor wound healing, the plate was placed in the IncuCyte S3 Live-Cell Analysis System (Essen BioScience) and images were acquired every hour. Data analysis was generated by the IncuCyte software using a set confluence mask to measure relative wound density over time.

Cell proliferation and apoptosis assay: Cell proliferation assays were performed using cell lines stably transfected with NucLight Red Lentivirus (Essen Bioscience) to accurately visualize and count the nucleus of a single cell. Cells were seeded on a 96-well plate and allowed to adhere for 24 hours. Test compounds were added at indicated concentrations. Caspase-3/7 Green Apoptosis Reagent (Essen Bioscience) was also added as a green fluorescent indicator of caspase-3/7-mediated apoptotic activity. To monitor cell proliferation and apoptosis over time, the plate was placed in the IncuCyte S3 Live-Cell Analysis System (Essen BioScience) and images were acquired every 2 hours. Data analysis was generated by the IncuCyte software using a red fluorescence mask to accurately count each cell nucleus and a green fluorescence mask to measure apoptosis over time.

Immunoprecipitation (IP): Cells were lysed in the Pierce™ IP Lysis Buffer purchased from Thermo Fisher Scientific and 1 mg of protein was allowed to bind overnight in 4° C. to agarose-conjugated antibodies (Santa Cruz Biotechnology): ITGB4 (Cat #: sc-13543 AC), FAK (Cat #: sc-271195 AC), PXN (Cat #: sc-365379 AC). IP beads were washed 5 times with 1×RIPA buffer and denatured in 2× reducing sample buffer at 95° C. for 5 minutes. Western blots according to aforementioned protocol were performed to determine IP results.

3D spheroid assay: 3D spheroid experiments were performed using cell lines stably transfected with NucLight Red Lentivirus (Essen Bioscience) to visualize red fluorescence as an indicator of cell viability. Cells were seeded on a 96-well ultra-low attachment plate and allowed to form spheroids overnight. Drug treatment was added as indicated along with Cytotox Green Reagent (Essen BioScience), used as a green fluorescence indicator of cell death due to loss of cell membrane integrity. To monitor cell proliferation and apoptosis over time, the plate was placed in the IncuCyte S3 Live-Cell Analysis System (Essen BioScience) and images were acquired every 2 hours. Data analysis was generated by the IncuCyte software using a red fluorescence mask to accurately measure intensity and area of red fluorescence, indicating spheroid viability and a green fluorescence mask, indicating cell death.

Cell cycle analysis: H2009 cells were harvested and pelleted after 72 hours following siRNA transfection. Ice cold 70% ethanol was added to the pellet with mild vortexing to fix the cells. The fixed cells were kept at 4° C. for PI staining. FxCycle™ PI/RNase Staining solution from Invitrogen was used for staining the DNA according to the manufacturer's protocol prior the FACS analysis. Univariate model of Watson (Pragmatic) was used for cell cycle analysis.

Confocal microscopy: 3D spheroids were seeded and imaged in 96-well clear ultra-low attachment microplates (Corning) using Zeiss LSM 880 confocal microscope with Airyscan at the Light Microscopy/Digital Imaging Core Facility at City of Hope. Images were processed using ZEN software and analyzed using ImageJ (Schneider, C. A.; Rasband, W. S. & Eliceiri, K. W. (2012), “NIH Image to ImageJ: 25 years of image analysis”, Nature methods 9(7): 671-675, PMID 22930834).

Seahorse XF Cell Mito Stress Test metabolic assay: Cells were seeded in complete growth medium on a Seahorse XF Cell Culture Microplate (Agilent Technologies, Santa Clara, CA, USA) to reach 90% monolayer confluence by the next day. One day prior to assay, 5 μM cisplatin was added for 24 hours. On the day of the assay, mitochondrial inhibitor compounds were added to injection ports of the XFe96 FluxPak sensor cartridge at a final concentration of: oligomycin 1 μM, FCCP 1 μM, rotenone/antimycin A 1 μM each. Culture medium was changed to assay medium: Seahorse XF RPMI medium supplemented with 1 mM sodium pyruvate, 2 mM L-glutamine, and 10 mM glucose. After completion of assay, cells were immediately stained with Hoechst dye and imaged using BioTek. Images were analyzed with QuPath (Bankhead, P. et al. (2017). QuPath: Open source software for digital pathology image analysis. Scientific Reports. doi.org/10.1038/s41598-017-17204-5) to obtain number of cells in each well and normalize data according to cell number.

ROS production assay: Cells were seeded in a 96-well plate and placed in an incubator at 37° C. for 72 hours. 50 μl of medium from each well was transferred to another 96-well plate to measure ROS production with ROS-Glo™ H₂O₂ Assay (Promega, Madison, WI, USA). Remaining plate with cells were used to perform CellTiter-Glo® Luminescent Cell Viability Assay (Promega) to normalize ROS data to number of viable cells. Luminescence was measured using a Tecan Spark 10M multimode microplate reader.

γH2AX foci staining and analysis: Cells were seeded (50,000 cells/well) on glass cover slips coated with 0.1% gelatin (Millipore) in a 12-well plate. Next day, 5 μM cisplatin was added for 24 hours. Cells were fixed in 4% formaldehyde for 30 minutes at room temperature and blocked. Primary antibody against γH2AX (Cell Signaling Technology) was incubated in 4° C. overnight. Then secondary antibody was incubated for 2 hours at room temperature. Cover slips were mounted on glass slides and imaged with Zeiss LSM 880 confocal microscope at the Light Microscopy/Digital Imaging Core Facility at City of Hope. Using QuPath (Bankhead, P. et al. (2017). QuPath: Open source software for digital pathology image analysis. Scientific Reports. doi.org/10.1038/s41598-017-17204-5), green fluorescent subcellular particles were counted in each nucleus to obtain γH2AX foci count per cell.

Exosome isolation and analysis: 200 μl of serum from patient or healthy donor was taken and diluted with PBS to 5 ml. The diluted serum was filtered through 0.22-micron syringe filter and ultra-centrifuged at 90,000×g for 90 minutes. The supernatant was decanted, and the pellet was washed and centrifuged in 15 ml of PBS as before. The PBS was decanted, and pellet was suspended in 50 μl of 1×RIPA buffer containing protease inhibitors for 30 minutes on ice and transferred to 1.5 ml tube for quantification and denaturation as mentioned above.

Chromatin Immunoprecipitation: Briefly, five million formaldehyde-fixed cells were lysed in 200 ul of SDS lysis buffer and diluted to 2 ml in ChIP dilution buffer in the presence of protease inhibitors. Lysates were sonicated using Bioruptor PICO for 3 cycles and each cycle has 10 repeats of 30 seconds pulse and 30 seconds break. Lysates were precleared in salmon sperm DNA and protein A agarose by centrifugation. Prior to addition of antibody, 10% of the lysate was used for input and the remaining lysate was divided into two equal parts, one for IgG control and other for H3K27 acetylated antibody from Diagenode. Downstream processing of the chromatin bound antibody was done as per the manufacturer's protocol for EZ-magna ChIP A/G (Millipore, Temecula, CA). The extracted DNA was used for SYBR green based qPCR assay using the primers sequences Upstream USP1R-5′-AGGTTCACAGCATTCTCAATCC-3′ (SEQ ID NO:2), Upstream USP1F-CAGTGCCTGTGAAACTTTGGA (SEQ ID NO:3), Promoter USP1F-CTCAGCTCTACAGCATTCGC (SEQ ID NO:4) and Promoter USP1R-GGCCATCCAATGAGACAAGG (SEQ ID NO:5). The data was analyzed based on the percentage of input.

In-silico prediction of paxillin conformational ensemble: Using in-silico modeling and enhanced sampling MD simulations, the conformational ensemble of Paxillin N-terminal domain between LD2 and LD4 bound to FAK was predicted. Starting from the crystal structures of FAK bound to the LD2 and LD4 motifs of Paxillin (pdb IDs 1OW8 and 1OW7), the disordered region of Paxillin (˜110 residues) between LD2 and LD4 as a random coil was modeled using Modeller. The N and the C termini of Paxillin and FAK were capped by the acetyl and N-methyl acetamide groups respectively. This structure was subjected to temperature replica exchange simulations (REMD) using the AMBER ff14SBonlysc force-field and the implicit solvation model GBneck2, as recommended in the AMBER16 manual. 26 replicas were used between 280K-480K and the individual temperatures were chosen to maintain an exchange success rate of 20%. To maintain structural integrity when subjected to high temperature during REMD, the backbone atoms of the helical regions of FAK and the bound LD2 and LD4 domains of Paxillin were position-restrained with a force of 5 kcal/mol. During REMD, the temperature was maintained using the Langevin thermostat with a collision frequency of 1.0. To accelerate the MD, the system was subjected to hydrogen mass repartitioning, which allowed a 4 femtosecond timestep to be used. The replicas were initially minimized using the conjugate gradient method followed by gradual heating to their respective temperatures over 1 ns. The REMD simulation was carried out for 1.15 μs for each replica, followed by clustering of the lowest temperature conformations by the Paxillin Ca atoms using hierarchical agglomerative clustering. The representative structures from the top 3 most populated clusters were used for virtual screening.

Virtual ligand screening to identify small molecule inhibitors of Paxillin-FAK interaction: The top Paxillin conformations obtained from REMD were scanned for druggable pockets using the program FindBindSite (FBS). The top ranked pockets in each Paxillin conformation were further screened by proximity to the FAK interface. In total, 4 pockets among 3 Paxillin conformations were selected for virtual screening. The protein structures were prepared using Maestro (Schrodinger™, LLC). The SelleckChem FDA-approved drug library containing approximately 1400 compounds was then docked to each pocket separately using Glide standard precision. The ligand library was prepared using the LigPrep module of Maestro and all possible protonation states at neutral pH were generated. During docking, the protein atoms were scaled by 0.8 and 10 docked poses per ligand were retained. Next, the best docked pose for each ligand was selected by Glide score and was optimized by reassigning the side chains within 5 Å of the docked ligand using Prime followed by minimization of the entire complex using MacroModel (Schrodinger™, LLC). Using MacroModel, a crude binding energy score was generated for each docked complex by subtracting the sum of individually solvated protein and ligand energies from the energy of the solvated complex. This score was used to select the top 50 ligands from each binding pocket, which were then subjected to thorough optimization and binding free energy calculation using the MMGBSA method in PrimeX. The top 10 compounds by binding free energy from each binding pocket were selected for experimental testing.

Fluorescence-activated cell sorting (FACS) and analysis: Cells were trypsinized and resuspended (5 million) in PBS with 2% FBS. Cells were stained with ITGB4 antibody conjugated to Alexa Fluor® 488 (5 μl/1 million cells) (R&D Systems, Minneapolis, MN, USA) and Propidium Iodide Ready Flow™ Reagent (1 drop/1 million cells) (Invitrogen) for 30 minutes at 4° C. The Analytical Cytometry Core Facility at City of Hope carried out and assisted all FACS sorting and analysis experiments. Gates were set to sort cell populations having low 10% and high 10% expression of ITGB4 using the FACSAria™ Fusion (BD Biosciences, San Jose, CA, USA). Sorted cells were immediately cultured in 12-well plates and treated with cisplatin (1 μM) for 48 hours. Then, equal number of untreated and treated cells were collected and stained with same reagents as above. FACS analysis was performed to determine shifts in cell population using the Attune NxT Flow Cytometer (Invitrogen).

Mathematical modeling: Bifurcation diagram was obtained MATCONT (cite: dl.acm.org/citation.cfm?id=779362). Next, Random circuit perturbation (RACIPE) algorithm was run on the two-node network—ITGB4/miR-1-3p. The continuous gene expression levels were obtained as output with randomly chosen parameters for the regulatory links. The algorithm was used to generate 100,000 mathematical models, each with a different set of parameters for the following ODEs:

$$u = {C}_{u} {H}^{s} ( l , {l}_{{u}^{′}}^{0} , {n}_{lu} , {λ}_{lu}^{-} ) - {k}_{u} u \\ l = {G}_{l} {H}^{s} ( u , {u}_{{l}^{′}}^{0} , {n}_{ul} , {λ}_{ul}^{-} ) - {k}_{l} l$$

where, u denotes miR-1-3p and l denotes ITGB4. Gu and Gl are the maximum production rates of miR-1-3p and ITGB4 respectively. And, ku and kl are their innate degradation rates respectively.

Additional equations are provided as follows:

$${μ}^{*} = {g}_{μ 3 p} {H}^{s} ( l , {λ}_{l , μ3 p )} - {m}_{l} {{Y}_{}}_{μ} ( {μ}_{3 p} ) - {k}_{μ3 p} {μ}_{3 p} \\ {m}_{l}^{*} = {g}_{ml} {H}^{s} ( C , {λ}_{c , ml} ) - {m}_{l} {Y}_{m} ( {μ}_{3 p} ) - {k}_{ml} {m}_{l} {l}^{*} = {g}_{l} {m}_{l} L ( {μ}_{3 p} ) - {k}_{l} l$$

where H^{S} is the shifted Hill function, defined as H^{S} (B,λ)=H⁻(B)+λH+(B), H⁻(B)=1/[1+(B/B₀)^{nB}], H⁺(B)=1-H⁻(B) and λ is the fold change from the basal synthesis rate due to protein B. λ>1 for activators, while λ<1 for inhibitors.

The total translation rate:

$${m}_{l} L ( {μ}_{3 p} ) = m {\sum }_{i = 0}^{n} {l}_{i} {C}_{n}^{i} {M}_{n}^{i} ( μ )$$

The total mRNA active degradation rate:

$${m}_{l} {Y}_{m} ( {μ}_{3 p} ) = m {\sum }_{i = 0}^{n} {γ}_{{m}_{i}} {C}_{n}^{i} {M}_{n}^{i} ( μ )$$

The total miR active degradation rate is

$${m}_{l} {Y}_{μ} ( {μ}_{3 p} ) = m {\sum }_{i = 0}^{n} {γ}_{{μ}_{i}} {C}_{n}^{i} {M}_{n}^{i} ( μ )$$

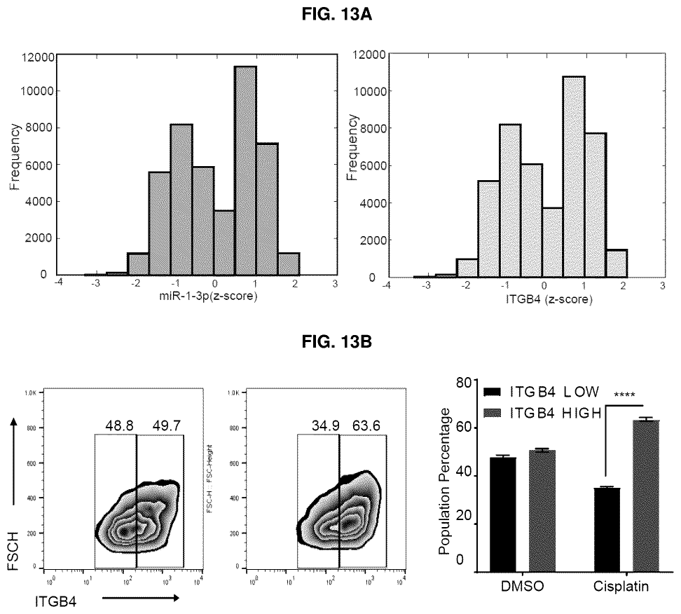

Parameters used in FIG. 13A are shown in Table 1 below:

TABLE 1

| | | | | |
|---|---|---|---|---|
| n (# of miR binding sites) | 0 | 1 | 2 | 3 |

| | | | | | |
|---|---|---|---|---|---|
| L only | lᵢ | 1.0 | 0.5 | 0.2 | 0.02 |
| Y only | γₘᵢ (Hour⁻¹) | | 0.3 | 1.5 | 7.5 |
| Both, | lᵢ | 1.0 | 0.6 | 0.3 | 0.1 |
| L stronger | γₘᵢ (Hour⁻¹) | | 0.04 | 0.2 | 1.0 |
| — | γₘᵢ (Hour⁻¹) | | 0.005 | 0.05 | 0.5 |

The parameters for microRNA-mediated dynamics were estimated from our previous models for microRNA-mediated regulation of EMT (Lu et al. 2013), shown in Table 2 below.

TABLE 2

| Parameter | Value |
|---|---|
| k_{μ3p} | 0.05 |
| (hour⁻¹) | |
| kₘₗ | 0.5 |
| (hour⁻¹) | |
| k_{I} | 0.1 |
| (hour⁻¹) | |
| g_{μ3p} | 2900 |
| (molecules/hour) | |
| gₘₗ | 30 |
| (molecules/hour) | |
| g_{I} | 100 |
| (hour⁻¹) | |
| I⁰_{μ3p} | 6000 |
| (molecules) | 10000 |
| μ3p⁰ | |
| (molecules) | 250000 |
| C⁰ₘₗ | |
| (molecules) | |
| n_{I, μ3p} | 3 |
| n_{μ3p} | 6 |
| n_{C, ml} | 3 |
| λ_{I, μ3p} | 0.3 |
| λ_{C, ml} | 10 |

### Example 2: Upregulation of Both PXN and ITGB4 but not PXN Alone Correlates with Cisplatin Resistance

To identify the genes upregulated in cisplatin resistance in LUAD cell lines, GSEA analysis was performed on RNAseq data from the Molecular Signatures Database v6.2 (MSigDB), a collection of annotated gene sets (Broad Institute) using the Gene Set Enrichment Analysis (GSEA) software. GSEA is a computational method that determines whether an a priori defined set of genes shows statistically significant concordant differences between two biological states. A set of eleven genes that were upregulated in cisplatin-resistant non-small lung cancer cells compared to sensitive cells were identified (Table 3).

TABLE 3

| Original | Entrez | Gene | |
|---|---|---|---|
| Member | Gene Id | Symbol | Gene Description |
| AF010316 | 9536 | PTGES | Prostaglandin E Synthase |
| AF019770 | 9518 | GDF15 | Growth Differentiation Factor 15 |
| J04164 | 8519 | IFITM1 | Interferon Induced Transmembrane Protein 1 |
| M29366 | 2065 | ERBB3 | V-erb-b2 ErythroblasticLeukemia Viral Oncogensis |
| M29870 | 5879 | RAC1 | Ras-related C3BotulinumToxin Substrate 1 |
| M33882 | 4599 | MX1 | Myxovirus(Influenza Virus) Resistance1 |
| S80437 | 2194 | FASN | Fatty Acid Synthase |
| U09579 | 1026 | CDKN1A | Cyclin-dependent Kinase Inhibitor 1A |
| U14588 | 5829 | PXN | Paxillin |
| X53587 | 3691 | ITGB4 | Integrin, Beta 4 |
| X74295 | 3679 | ITGA7 | Integrin, Alpha 7 |

Four of these eleven genes namely, PXN, ITGB34, ITGA7, and Rac Family Small GTPase 1, are constituents of the FA complex activation, formation, and downstream signaling pathways. Therefore, to verify the correlation of these genes with cisplatin resistance, five LUAD cell lines that harbored wild type (WT) KRAS and five that carried a mutant (MT) version of KRAS were randomly selected (Table 4), treated with 10 μM cisplatin for 72 hours, and cell viability was determined using the CCK-8 viability assay.

TABLE 4

| | | Standard | Relative | |
|---|---|---|---|---|
| | Estimate | Error | Risk | P-Value |
| gender[1] | −0.04918 | 0.154775 | 0.952008 | 0.750665 |
| stage[S2] | 0.738091 | 0.194204 | 2.09194 | 0.000144 |
| stage[S3] | 1.17346 | 0.192492 | 3.23316 | 1.09E−09 |
| stage[S4] | 1.31524 | 0.284479 | 3.72563 | 3.78E−06 |
| age | 2.36E−05 | 2.14E−05 | 1.00002 | 0.271914 |
| ENSG00000089159* | 0.000156 | 3.03E−05 | 1.00016 | 2.47E−07 |
| ENSG00000132470 | | | | |

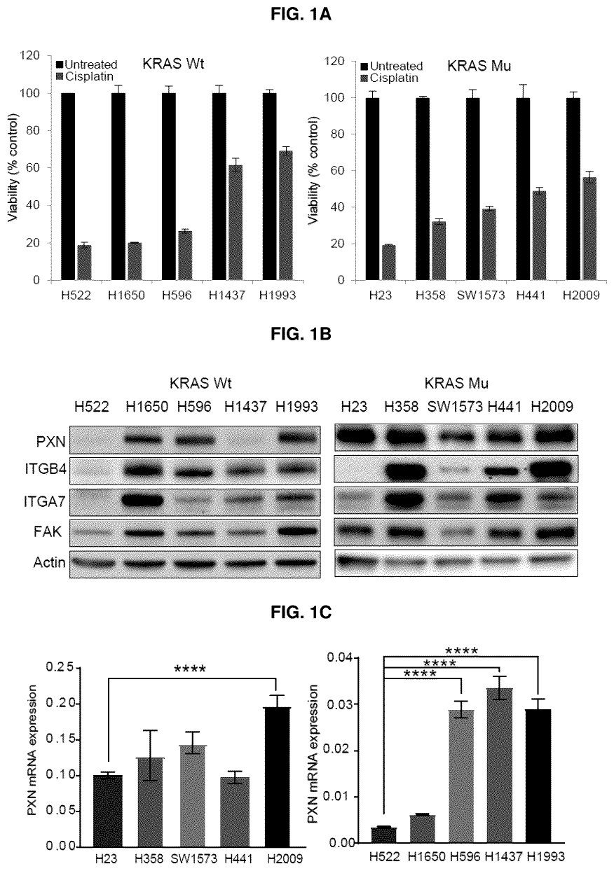

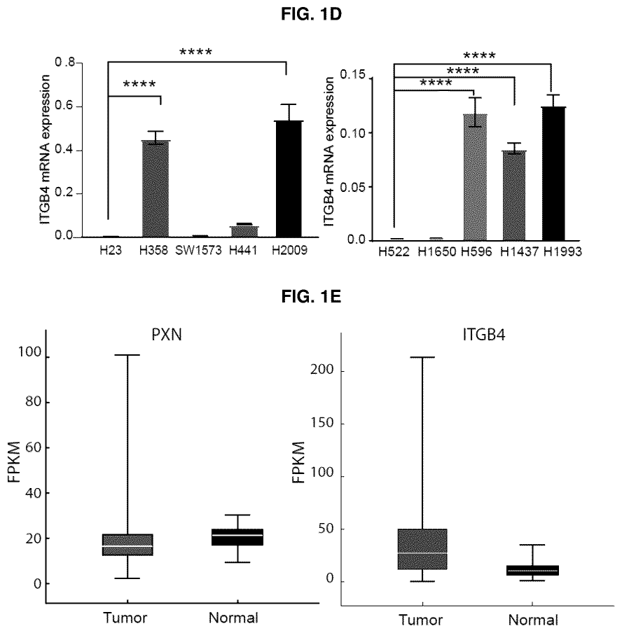

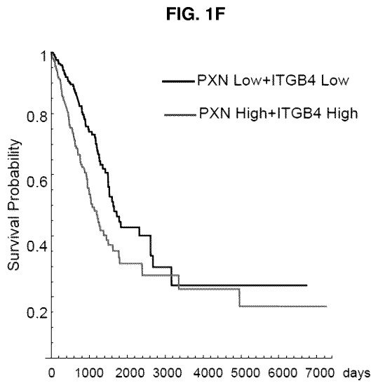

Among the KRAS WT cell lines, H1437 and H1993 were significantly more resistant while amongst the KRAS MT cell lines, H441 and H2009 were relatively more resistant than the other three cell lines (FIG. 1A).

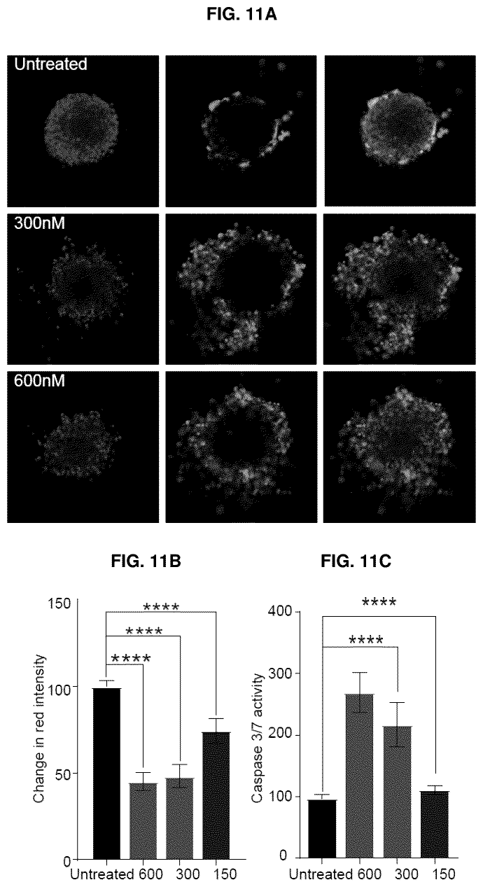

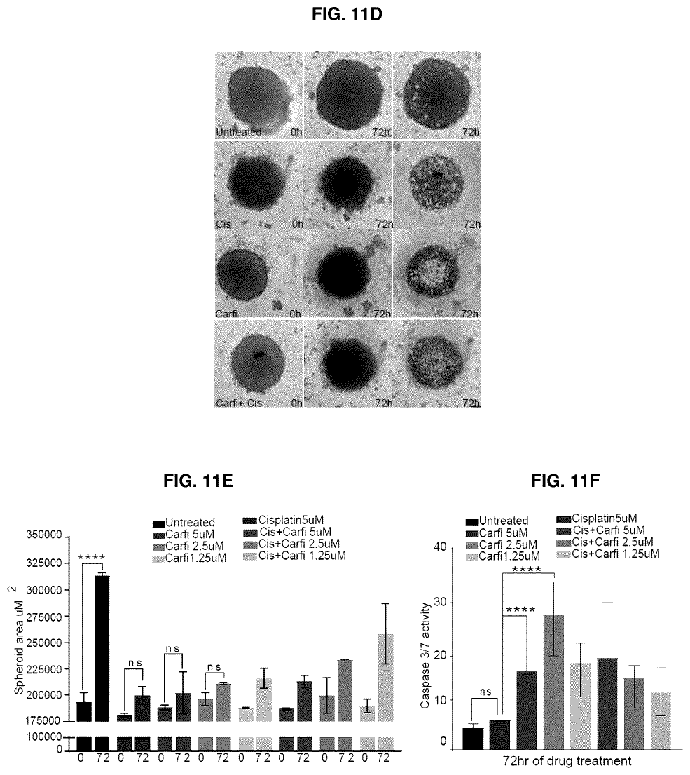

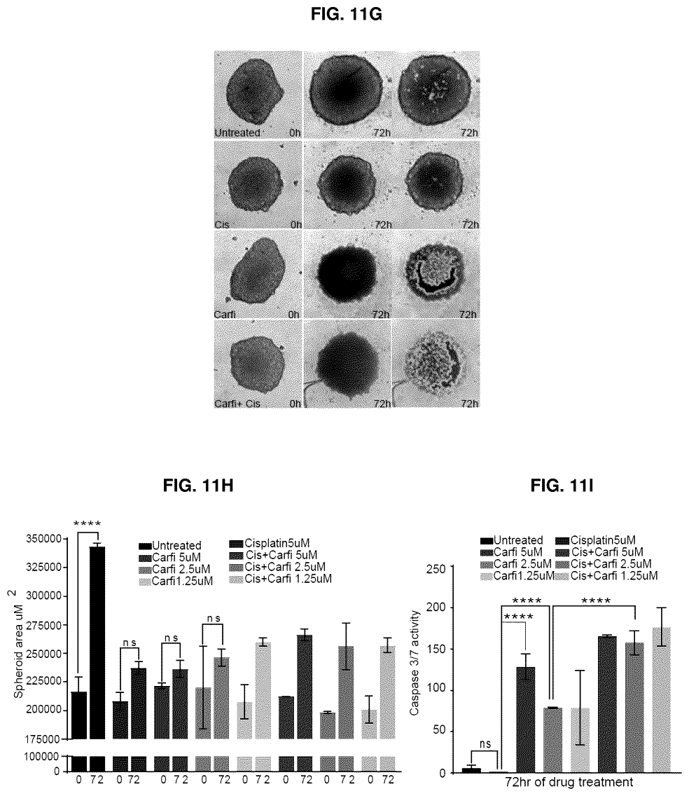

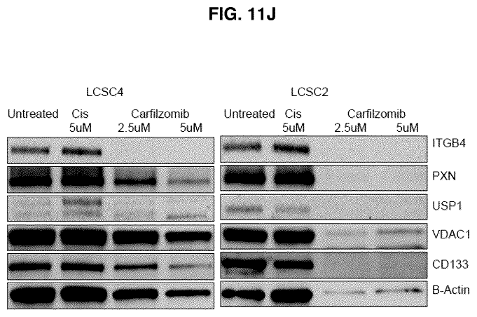

Next, the protein expression of PXN, ITGB4, ITGA7, and FAK in these cell lines was determined by immunoblotting. Rac1 expression was undetectable in these cell lines. However, PXN and FAK were expressed in all the cell lines tested regardless of whether they were cisplatin-sensitive or resistant (FIG. 11B). While the expression of ITGB4 and ITGA7 was variable among the cell lines, ITGB4 expression was high in the two cisplatin-resistant cell lines, H2009 and H1993. The mRNA expression patterns encoding these two proteins were analyzed in these cell lines and it was confirmed that the mRNA was consistently upregulated for both PXN and ITGB4 in the H2009 and H1993 cell lines (FIGS. 1C & 1D).

### Example 3: Co-Expression of PXN & ITGB4 Correlates with Poorer Prognosis in LUAD Patients

Since LUAD cell lines overexpressing PXN appeared sensitive to cisplatin treatment when they did not express ITGB4, it is possible that there is an interaction between PXN and ITGB4. Gene expression profiles of the patients diagnosed with “Lung Adenocarcinoma” were extracted from The Cancer Genome Atlas (TCGA) database and analyzed for expression of ITGB4 and PXN (FIG. 1E). The gene-level expression values, FPKM, were analyzed according to the Cox proportional hazards model: Survival˜Sex+Stage+Age+PXN*ITGB4. Stage and PXN*ITGB4 showed significant contribution to the model. The patients were separated into two groups by the median value of the PXN*ITGB4 product. The Kaplan-Meier curves showed clear survival difference between the two groups and the log rank test showed significant differences between the two curves (p=0.000089) (FIG. 1F).

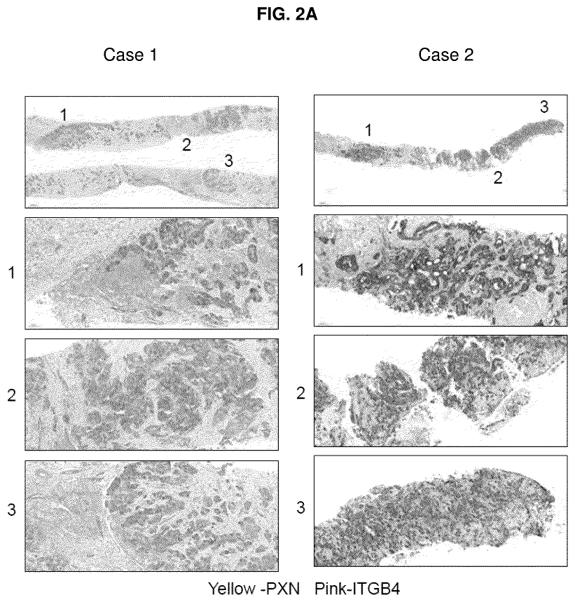

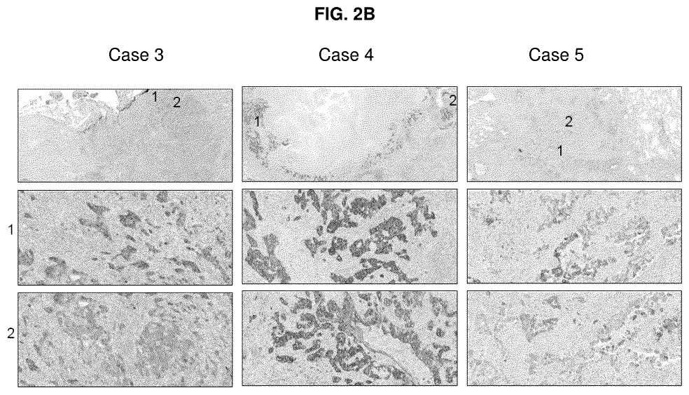

Immunohistochemistry on needle biopsy specimens obtained from 2 cases was performed. PXN staining was detected using yellow and ITGB4 with pink color. Thus, the coexpression of the t2 molecules would generate an orange or bright red color based on the expression level. Indeed, significant coexpression of ITGB4 and PXN was observed in various regions of the tumor (FIG. 2A). The case represented in FIG. 2A had weak expression of ITGB4 and coexpression generated an orange color. The second case had high expression of ITGB4 and for the same reason it generated a reddish pattern implying coexpression. The IHC staining was extended to 3 more cases. Variability in the expression of PXN and ITGB4 among cases was observed and within the sample as well. PXN was expressed in most of the region of the tumor but ITGB4 expression was sporadic and the level of ITGB4 expression was also variable among cases. Case 4 had high expression of ITGB4, Case 3 had intermediate expression and Case 5 had weak expression. The coexpression of PXN and ITGB4 (red or orange color) was limited to some regions of the tumor, emphasizing heterogeneity in their expression patterns (FIG. 2B). This observation indicates that cisplatin treatment favors selection of a subclonal population of tumor cells with coexpression of ITGB4 and PXN. Therefore, the role of ITGB4 in cisplatin resistance in those cell lines that had high expression of PXN was investigated.

### Example 4: Knocking Down ITGB4 Attenuates Proliferation and Migration of Cisplatin-Resistant Cells

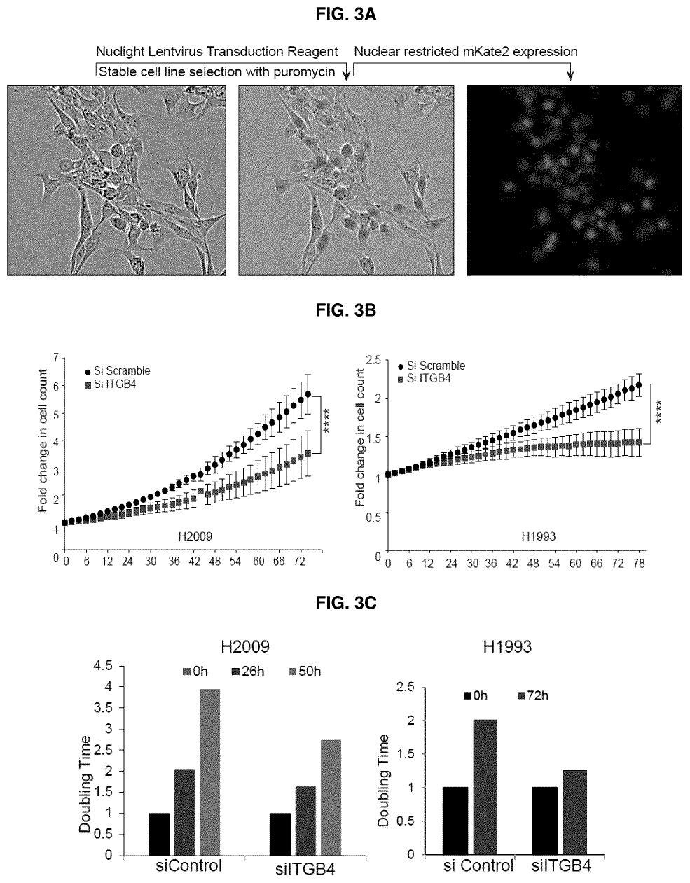

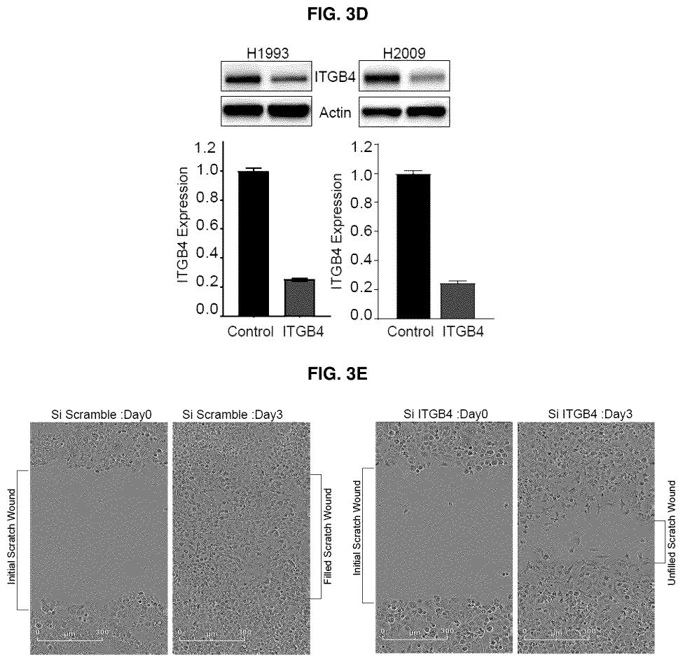

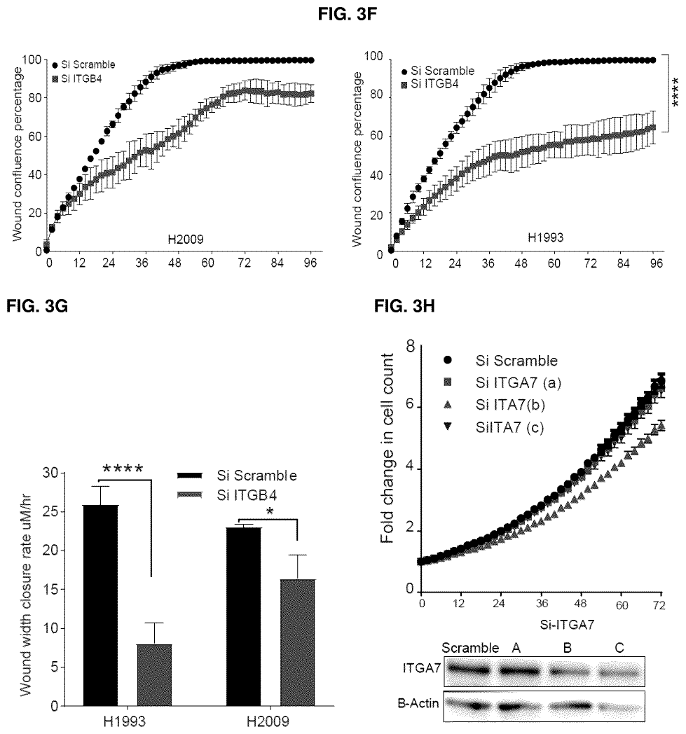

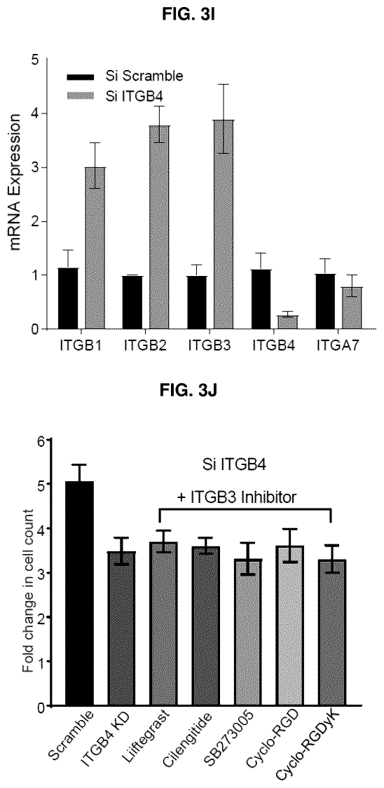

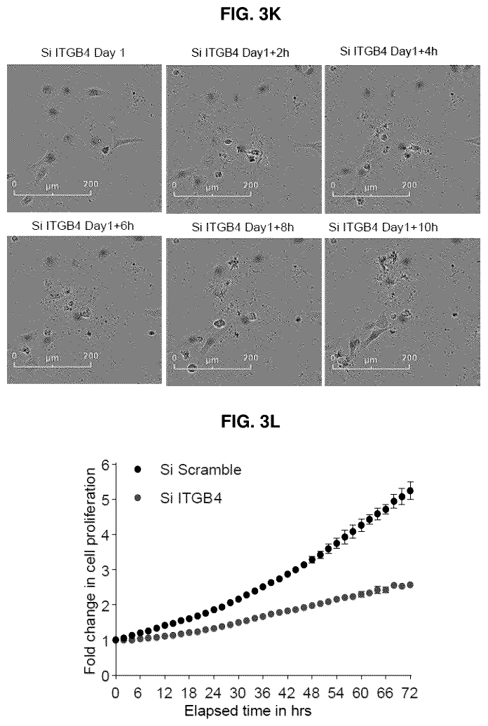

The effect of knocking down ITGB4 on cell proliferation was determined using H1993 and H2009 cells. Cells were transiently transfected with a control (scrambled) and ITGB4-specific siRNA, and the effect of silencing its expression on cell proliferation was determined using the IncuCyte Live Cell Imaging System (Essen Bioscience, Ann Arbor, MI). To facilitate live cell analysis, stable cell lines with nuclear expression of the red fluorescence protein (RFP) mKate2 were generated (FIG. 3A). Knocking down ITGB4 significantly attenuated their proliferation (FIG. 3B) and immunoblotting analysis using total cell lysates and qPCR analysis with RNA extracted from transfected cells confirmed a significant decrease in ITGB4 both at the protein and mRNA level (FIG. 3D). The doubling time of both cell types was analyzed. In the case of the H2009 cells, it was observed that by 50 hours post transfection, these cells showed a 4-fold increase in cell count, indicating a doubling time to be approximately 25 hours. However, in ITGB4 knocked down H2009 cells, the doubling time increased to 40 hours. Similarly, in H1993, knocking down ITGB4 led to only 0.2-fold increase in cell count by 72 hours, leading to an estimated doubling time of approximately 120 hours compared to 72 hours for control cells (FIG. 3C).

To discern the effect of knocking down ITGB4 on cell migration, H1993 and H2009 cells were transfected with the control or ITGB4-specific siRNA in 6-well plates and 12 hours post transfection, the cells were trypsinized and reseeded at high density in a 96-well plate. A scratch wound was generated using the WoundMaker from Essen Bioscience and wound healing was observed in real time using the IncuCyte Live Cell Imaging System (FIG. 3). While both cell lines transfected with the control siRNA achieved 100% confluency of the wound within 48 hours, H1993 and 2009 cells in which ITGB4 was knocked down reached ˜60% and ˜80% confluency, respectively; neither cell line reached 100% confluency even after 96 h (FIG. 3F). Next the rate of wound closure was estimated. The rate was determined by dividing initial wound width by the time taken to close the wound. In the case of the H1993 cells, the wound closure rate was ˜25 μm/h, and on knocking down ITGB4, the rate significantly dropped to <10 μm/h. Similarly, in the case of the H2009 cells, the wound width closure rate dropped from 22 μm/h to about 18 μm/h (FIG. 3G). Together, these results confirmed the significance of ITGB4 in modulating cell proliferation and migration in cisplatin-resistant LUAD cell lines. Integrin A7 was also knocked down in H2009 cell lines to validate its role in cell proliferation but no significant change was observed (FIG. 3H). Additionally, up regulation of expression of other integrin β forms in response to ITGB4 knockdown was checked and an increase in the expression of ITGB1, ITGB2, and ITGB3 at mRNA level was found (FIG. 3I). The effect of ITGB3 silencing in the ITGB4 knocked down cells was further tested but no significant difference in their proliferation was found (FIG. 3J).

### Example 5: Knocking Down ITGB4 Increases Sensitivity to Cisplatin Treatment

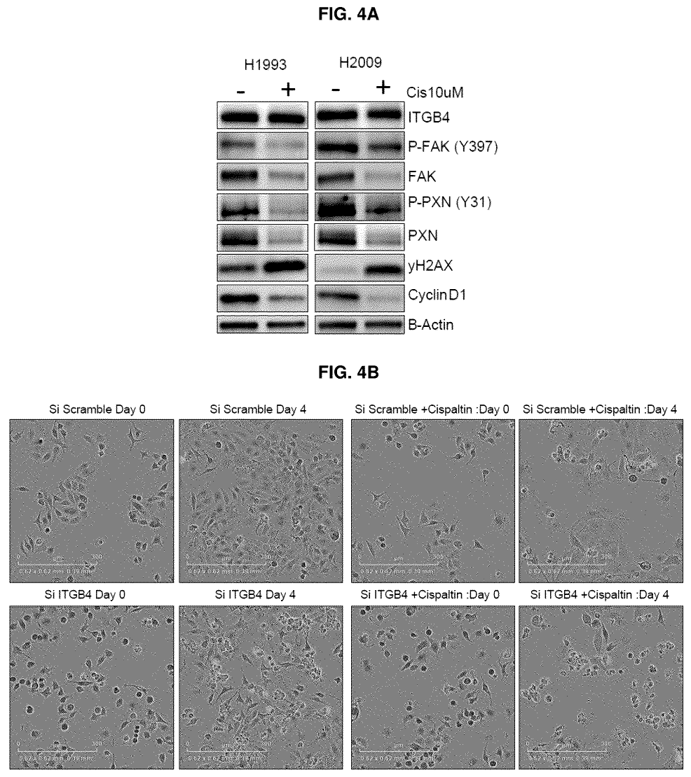

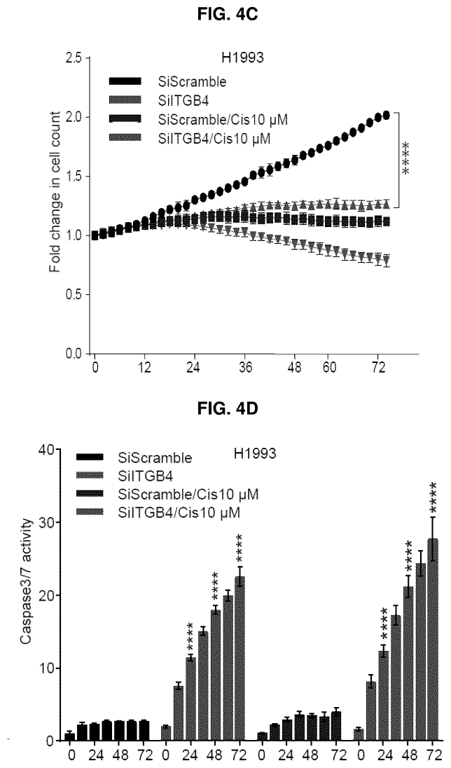

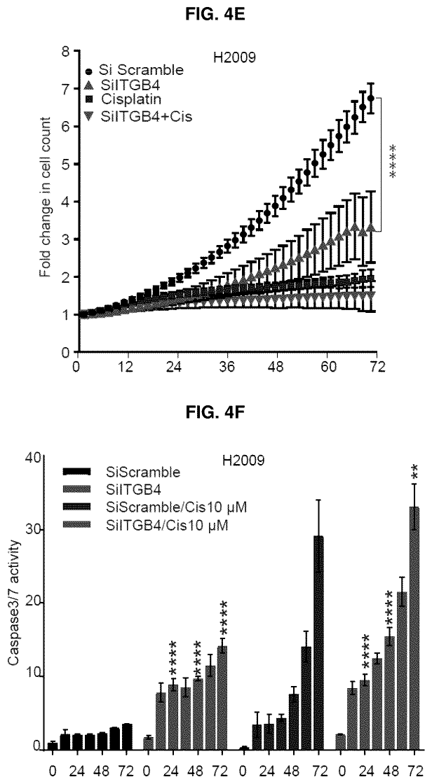

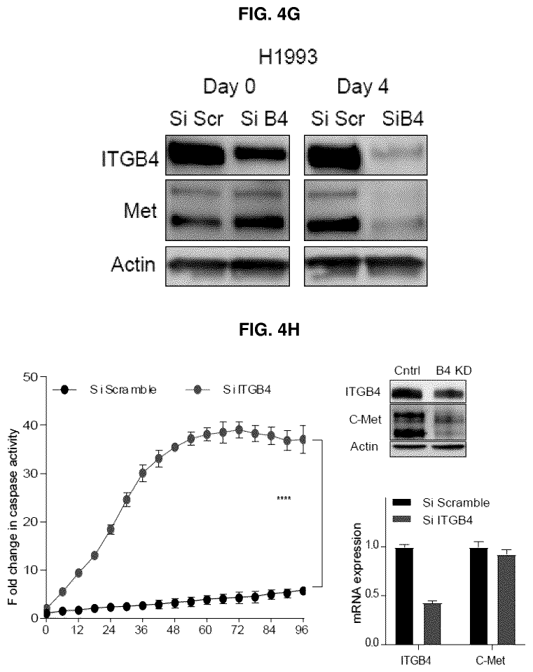

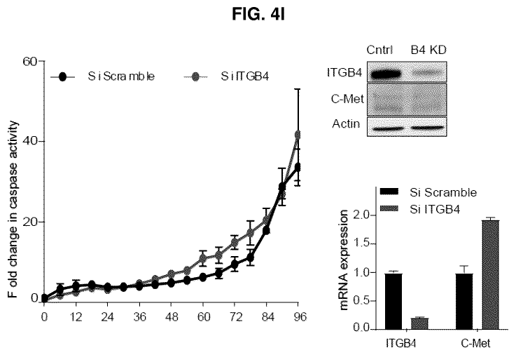

To determine whether the inhibition in cell proliferation associated with ITGB4 knockdown was due to enhanced apoptosis or reduced cell division (cytostatic), the Essen Bioscience Caspase-3/7 Green apoptosis assay in live cells was conducted. The NucView™633, a DNA intercalating dye was used to enable quantification of apoptosis over time. The dye is impermeable and non-toxic to cells but once the cell membrane is permeabilized by caspase-3/7, the dye enters the cell and gets activated due to change in pH leading to the generation of green fluorescence that is then detected in real time and quantified using the IncuCyte System (FIG. 4B).

To address this, H1993 and H2009 cells were treated with cisplatin for 3 days and the changes were analyzed by immunoblotting. A reduction in the expression of phosphorylated as well as total FAK and PXN was observed, but not in ITGB4 expression (FIG. 4A). Next, H1993 and H2009 cells were transiently transfected with ITGB4 siRNA and 24 hours post-transfection they were treated with 10 μM cisplatin and cell proliferation and caspase activity were determined. In H1993, ITGB4 knockdown, or cisplatin treatment, inhibited cell proliferation significantly and the fold change in cell count was ˜1 indicating a cytostatic effect. However, in response to cisplatin in ITGB4 knockdown cells, the fold change dipped to <1, alluding to a cytotoxic effect (FIG. 4C). Further, live caspase activity analyzed in the same cells showed a 5- to 6-fold increase in activity compared to control by 24 hours and by 72 hours, caspase activity increased to 20-fold. Addition of cisplatin to the ITGB4 knockdown cells had an additive effect but cisplatin treatment alone did not induce caspase activity, indicating the reduction in cell proliferation by cisplatin was cytostatic but that knocking down ITGB4 induced cytotoxicity (FIG. 4D). In KRAS MT H2009 cells, a similar trend was observed but the cell proliferation did not drop <1 and the induction of caspase activity by ITGB4 knockdown was weak. Knocking down ITGB4 induced caspase activity within 24 hours of seeding, whereas cisplatin treatment alone induced apoptosis in H2009 cell by 72 hours and the combination had an additive effect (FIGS. 4E and 4F). ITGB4 knockdown in H1650 cell line also inhibited cell proliferation and induced cell death. However, cell death was not due to caspase activation but appeared to be due to cell bursting which is seen in anoikis (FIGS. 3K and 3L). Together, these data underscore the role of ITGB4 in imparting cisplatin resistance in these two LUAD cell lines irrespective of their KRAS status.

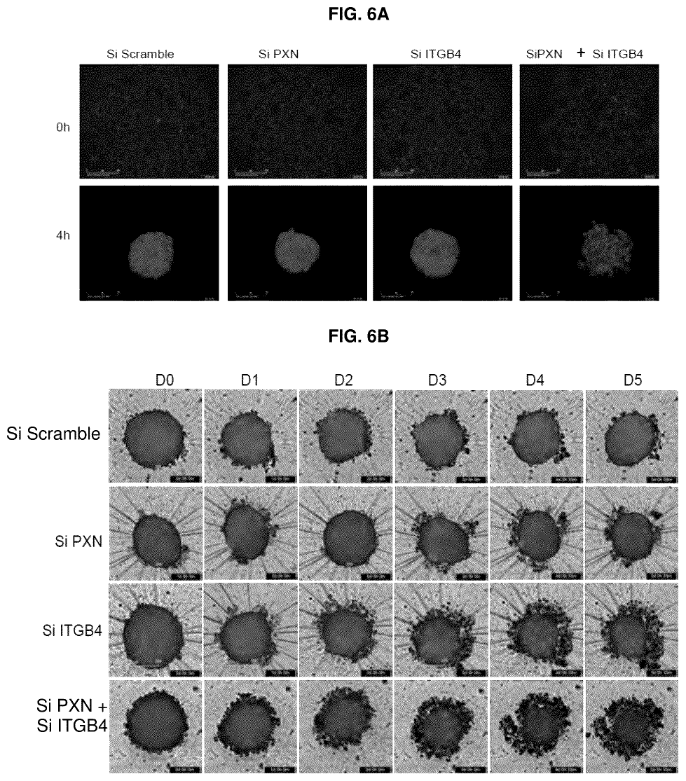

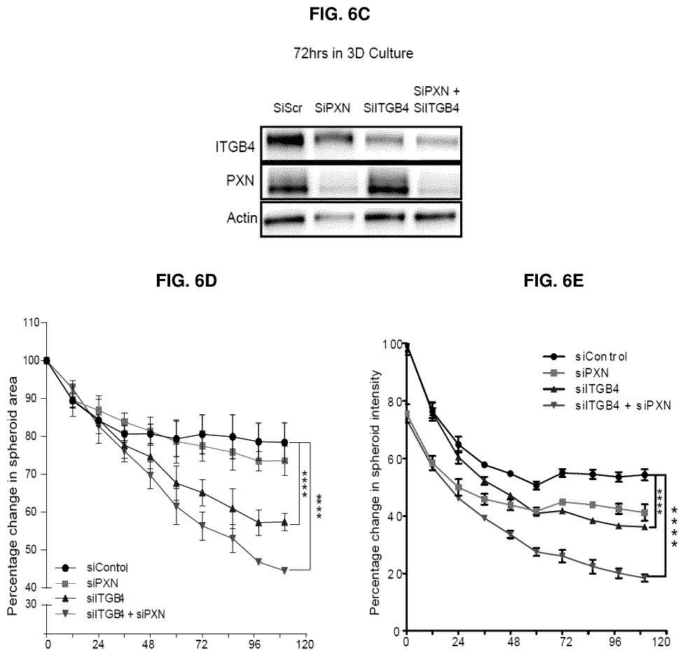

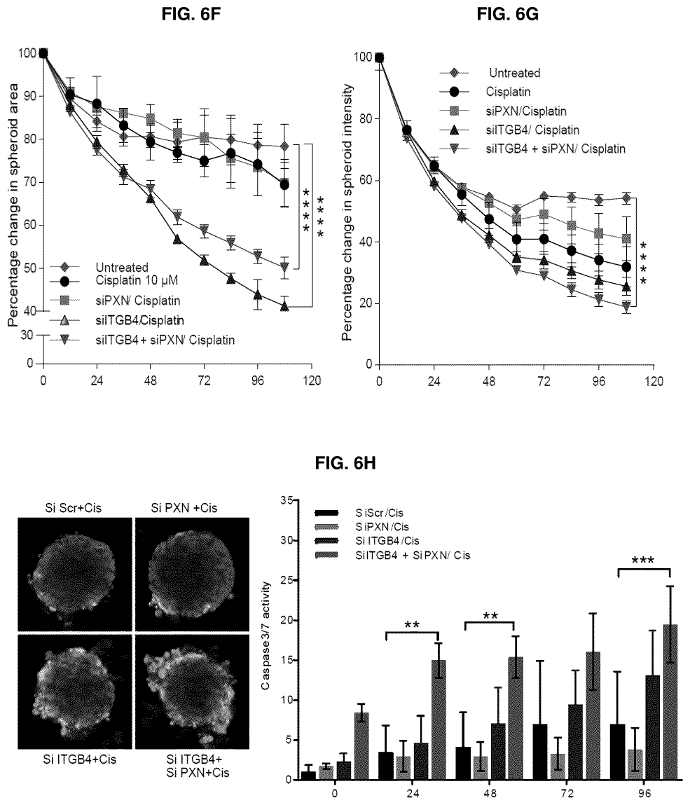

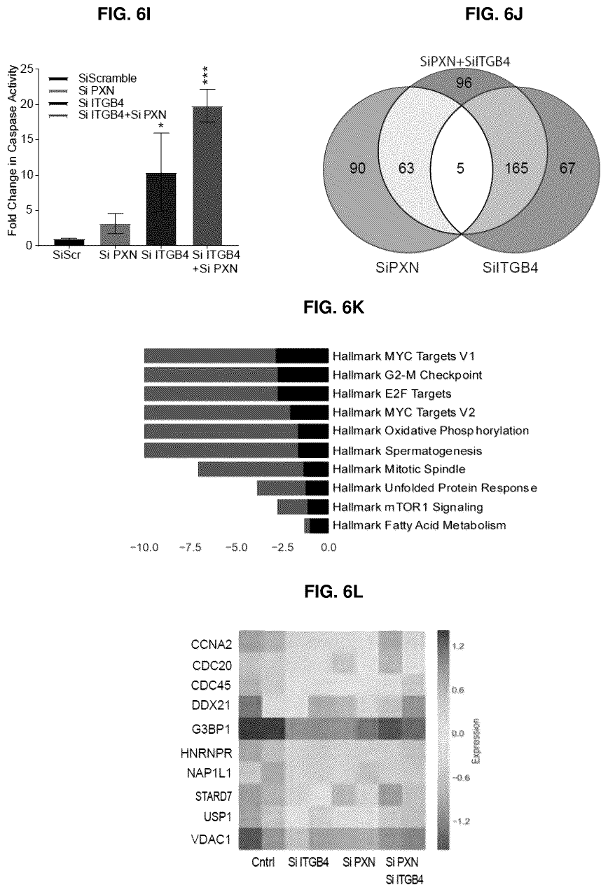

It has been shown that the cytoplasmic domain of ITGB4 acts as a scaffold for various kinases involved in activating the MAPK, PI3k or Akt pathways (Trusolino et al, 2001). MET Proto-Oncogene, Receptor Tyrosine Kinase (MET) is also one of the interacting partners of ITGB4 and is responsible for HGF-dependent phosphorylation and activation of ITGB4 (Trusolino et al, 2001). Of the two cell lines used in the present study, H1993 is known to harbor MET amplification. Thus, whether ITGB4 knockdown disrupts MET activity was investigated. Indeed, a decrease in MET expression in ITGB4 knocked down cells at the protein level was observed but there was no significant change in the mRNA expression, suggesting that ITGB4 may be involved in MET protein stability in these cells (FIG. 4G and FIG. 4H). However, in H2009 cells that do not carry MET amplification, MET expression was weak and no detectable decrease in MET expression was observed (FIG. 6A). These data suggested that the cells dependent on MET signaling (H1993) may be more sensitive to ITGB4 knockdown while cells independent of MET signaling (H2009) may be insensitive.

### Example 6: Treating Cells with ITGB4 Antibody or Knocking Down Both PXN and ITGB4 has a Synergistic Effect on Attenuating Cisplatin Resistance

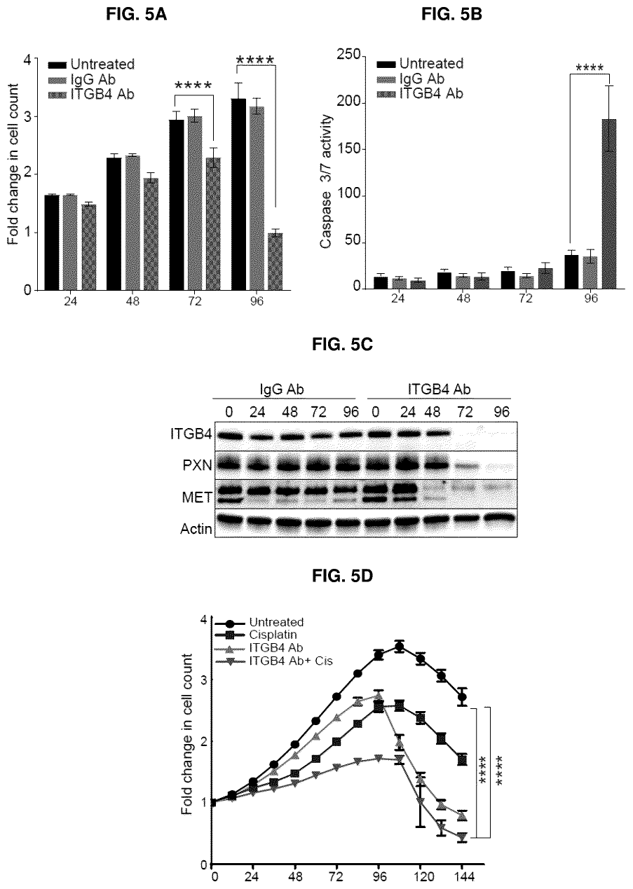

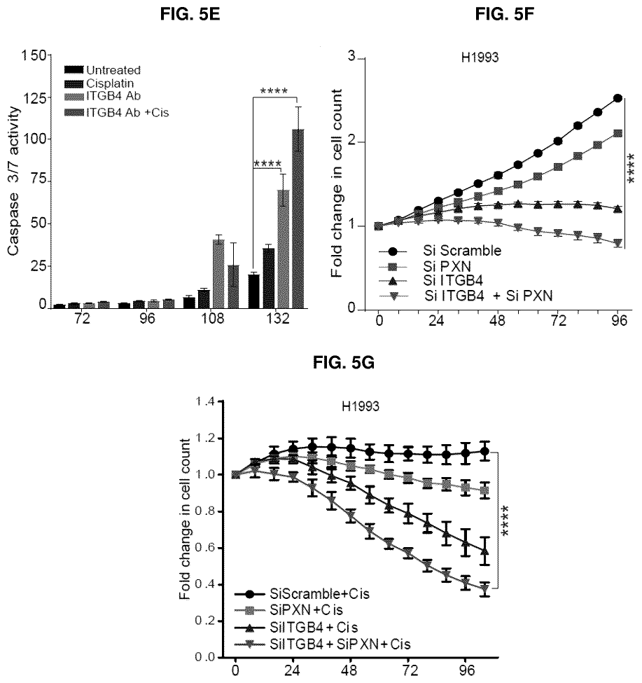

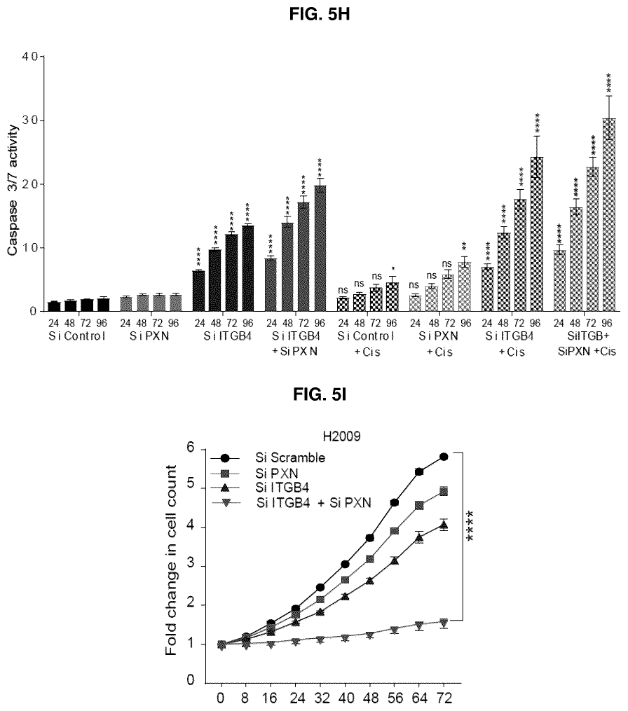

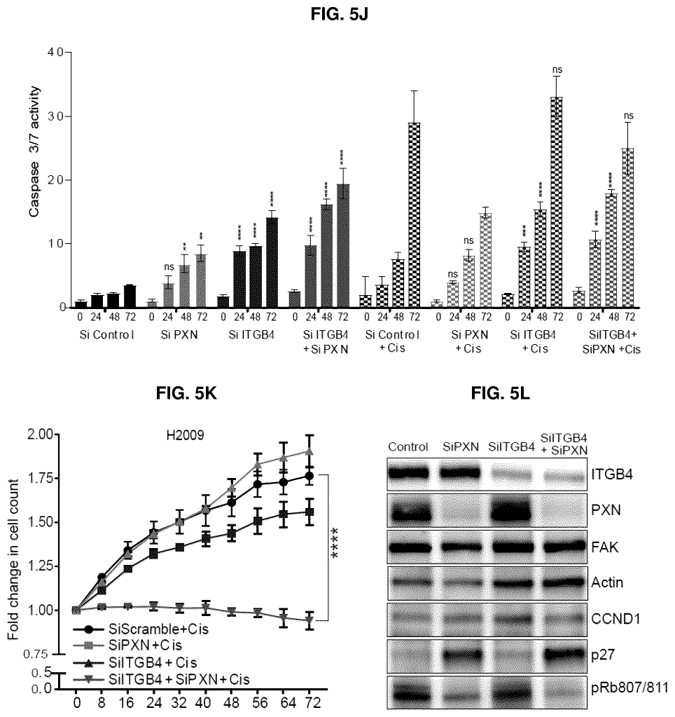

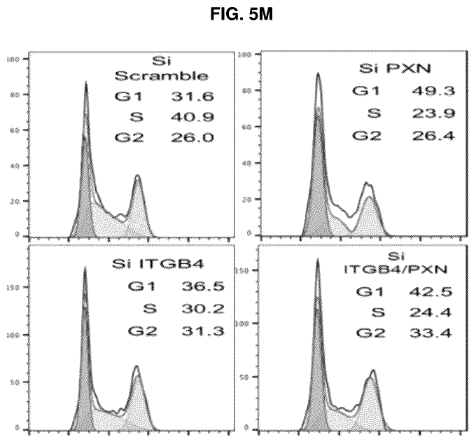

Having demonstrated that silencing ITGB4 expression can have a significant effect on response to cisplatin, whether blocking the ITGB4 extracellular epitope with an antibody would have a similar effect was tested. H1993 cells were first incubated with anti-ITGB4 antibody for 6 hours in ultra-low attachment tissue culture plates and then plated on tissue culture-treated plates. As expected, antibody-treated cells were unable to attach to the plate, indicating neutralization of ITGB4 epitopes by the antibody. The cells were allowed to grow for 72 hours and proliferation was determined using red fluorescence count. It was observed that antibody-treated cells proliferated slower than untreated or control IgG-treated cells. However, ITGB4 antibody treatment induced 5- to 7-fold higher caspase activity than untreated cells (FIGS. 5A and 5B). Furthermore, immunoblotting performed on whole cell lysates prepared from these cells showed a decrease in ITGB4 and PXN protein levels after 48 hours of culturing, which further declined steadily until 72 hours (FIG. 5C). In contrast, cells incubated with control IgG antibody in ultra-low attachment plates for the same period of time and re-plated in tissue-culture treated plates did not show any change in the ITGB4 protein level, suggesting that decrease in protein levels was due to anti-ITGB4 antibody treatment and not due to any difference in culturing conditions.

Next, the effect of combining the antibody and cisplatin treatments was investigated to determine potential synergy between the two modalities. Indeed, combining the two treatments showed an additive effect on cell proliferation and caspase-3/7 activity compared to either treatment alone. Furthermore, a synergistic effect was also observed even when a much lower dose (2.5 μM instead of 10 μM) of cisplatin was used (FIGS. 5D and 5E), suggesting that tumors expressing high ITGB4 can be treated with anti-ITGB4 antibody in combination with low dose of cisplatin, which in turn can reduce the undesirable toxicity associated with cisplatin.

Since cisplatin treatment caused changes in expression of FAK and PXN but not ITGB4 (FIG. 4A), and all three proteins are required for FA complex formation and activation, whether PXN knockdown alone can induce similar phenotype or PXN and ITGB4 double knockdown can synergistically change cell proliferation and survival rates was investigated. In H1993 cells, PXN knockdown inhibited cell proliferation by 15% compared to 50% inhibition by ITGB4 knockdown and 70% inhibition caused by double knockdown (FIG. 5F). Treating H1993 single and double knockdown cells with cisplatin induced cytotoxic effect; a drop in the fold change of cell count by 40% and 60%, respectively was observed compared to control (scramble siRNA) cells treated with cisplatin. However, cisplatin treatments of PXN knockdown cells had a weaker effect (FIG. 5G). The effect of knockdown on apoptosis was further tested and it was found that PXN knockdown did not activate caspase-3/7 but had an additive effect when knocked down together with ITGB4. Further, knocking down ITGB4 alone or together with PXN made the cells prone to cisplatin toxicity by inducing apoptosis (FIG. 5H). In the case of the H2009 cells, double knockdown strongly inhibited cell proliferation in presence and absence of cisplatin (FIGS. 51 and 5J). Again, double knockdown induced strong apoptosis within 24 hours compared to PXN or ITGB4 single knockdown. In the presence of cisplatin, double knockdown caused significant apoptosis even at early time points (FIG. 5K). Immunoblotting analysis confirmed knockdown of both ITGB4 and PXN (FIG. 5L) but surprisingly, PXN knockdown induced activation of p27 and a reduction in phosphorylation of the retinoblastoma protein Rb1, suggesting cell cycle inhibition. Indeed, cell cycle analysis following knocking down PXN or ITGB4 individually or together showed that knocking down PXN induced G1-S arrest, whereas knocking down ITGB4 resulted in G2-M arrest and the double knockdown inhibited both G1-S and G2-M phases (FIG. 5M), which may explain the synergistic phenotype.

### Example 7: Knocking Down PXN or ITGB4 Disrupts Spheroid Growth

To mimic conditions closer to in vivo, the effect of knocking down PXN or ITGB4 was discerned in spheroids (3D) formed from the cell lines used in 2D culture. H2009 cells engineered to express RFP (mKate2) were first transfected with scrambled or PXN/ITGB4-specific siRNA and then seeded in an ultra-low attachment round bottom plate for generating spheroids. Within 4 hours of seeding, spheroid formation in wells seeded with cells that were transfected with the control siRNA was observed but not in the cell transfected with ITGB4 and PXN siRNA (FIG. 6A). The double knockdown cells took longer time for forming spheroids. Once they formed spheroids, they were monitored for 5 days using IncuCyte live cell imaging system (FIG. 6B). The spheroid growth and survival were quantitated by monitoring dye intensity and area in real time. A reduction in spheroid area in double knock down cells by >40% compared to that seen in the spheroids formed by cells transfected with the scrambled siRNA was observed (FIG. 6D). Again, spheroids formed using the cells with the double knockdown showed decline in red fluorescence intensity and by day 4, the intensity was only ˜30% of the control (FIG. 6E). Additionally, the single and double knockdown spheroids were treated with cisplatin and it was observed that double knockdown spheroids were most sensitive followed by spheroids with only ITGB4 knocked down. However, PXN knocked down spheroids behaved similar to that of control (FIGS. 6F and 6G). By immunoblotting analysis, it was confirmed that spheroids continued to have diminished levels of ITGB4 and PXN by 72 hours post-transfection (FIG. 6C). To confirm that the reduction in spheroid size was due to cell death upon treatment with cisplatin, they were assayed for caspase-3/7 activity using the Essen Bioscience assay described above. A 20-fold higher caspase 3/7 activity was observed in double knockdown compared to Si Scramble by day 5 (FIG. 6I). Caspase activity in control spheroids and double knockdown spheroids treated with cisplatin was also analyzed and the double knockdown spheroids exhibited a strong synergistic effect (FIG. 6H). Considered together, the data from the 2D and 3D culture experiments revealed that the interaction between PXN and ITGB4 is important for modulating cisplatin resistance in these LUAD cell lines.

### Example 8: RNAseq Analysis Suggests that Multiple Pathways Converge in Cisplatin Resistance

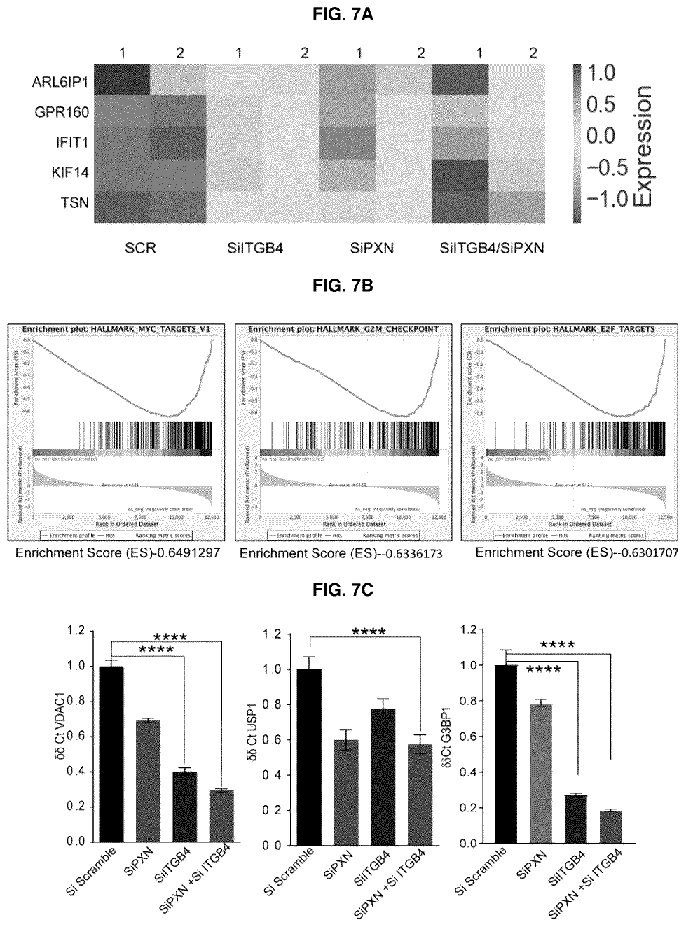

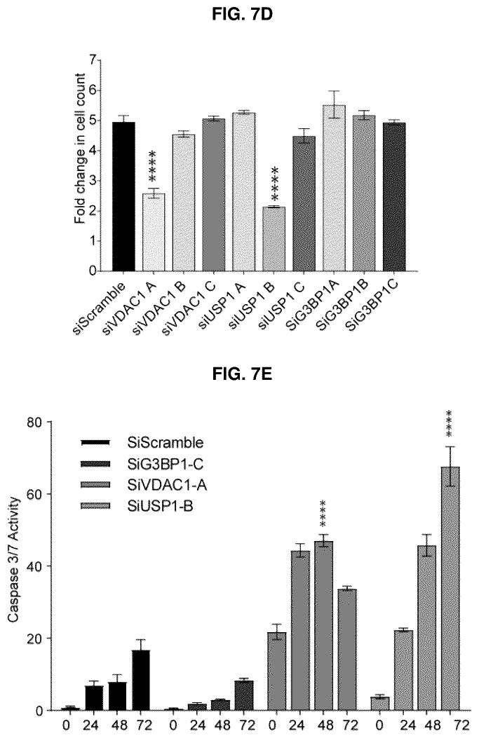

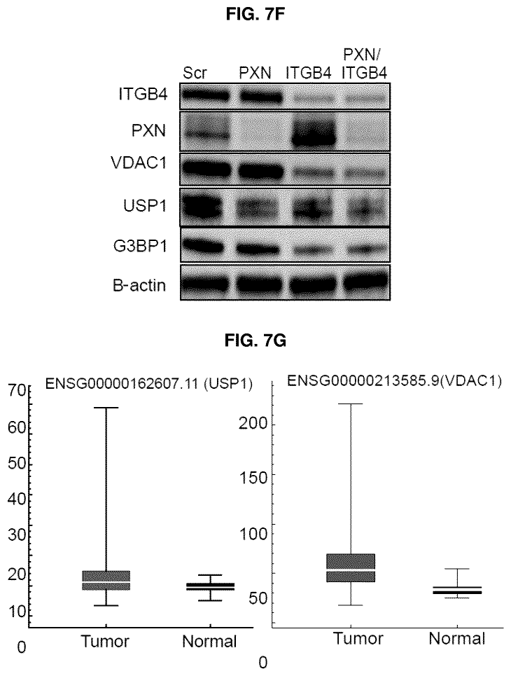

To identify the effect of knocking down ITGB4/PXN on the expression of the genes involved in signaling, the changes in global gene expression patterns were determined using RNAseq. RNA was extracted from both single and double knockdown cells 48 hours post siRNA transfection and total RNAseq was performed as described in Example 1. In all, 30 million reads were analyzed for each condition. In all, 237 genes were downregulated when ITGB4 was knocked down and 158 genes were downregulated upon knocking down PXN. In the case of the double knockdown, 329 genes that were downregulated were identified (FIG. 6J). Interestingly, 5 genes ARL6IP1, GPR160, IFIT1, KIF14 and TSN were common to both single and double knockdown samples. Of these, ARL6Ip1 is known to have anti-apoptotic role and KIF14 is known to control cell division, cell cycle progression, and apoptosis (FIG. 7A). Comparative GSE analysis of the RNAseq data between control and double knockdown showed significant enrichment of gene sets regulated by MYC (FIG. 6K), genes associated with G2-M checkpoint, and genes regulated by the E2-F transcription factor required for cell cycle progression (FIG. 7B). Myc is a proto-oncogene whose upregulation induces tumorigenesis. Therefore, the effect of ITGB4 and PXN in regulation of the MYC targets was explored. The top 10 genes contributing significantly to down regulation of the MYC pathways were interrogated (FIG. 6L). DAVID analysis was done on the unique 96 genes that were downregulated in only double knockdown cells (Table 5). DAVID analysis of the 206 genes were downregulated in double knockdown as well as PXN knockdown or ITGB4 knockdown, are listed in Table 6.

TABLE 5

| Category | Term | Fold Enrichment | Count | PValue |
|---|---|---|---|---|
| GOTERM_BP_DIRECT | GO: 0008355~regulation of transcription, DNA-templated | 2.232978723 | 17 | 0.0029 |
| GOTERM_BP_DIRECT | GO: 0003158~endothelium development | 65.85098039 | 2 | 0.0296 |
| GOTERM_BP_DIRECT | GO: 0000086~G2/M transition of mitotic cell cycle | 5.767969085 | 4 | 0.0314 |
| GOTERM_BP_DIRECT | GO: 0006351~transcription, DNA-templated | 1.717851662 | 17 | 0.0316 |
| GOTERM_BP_DIRECT | GO: 0015677~copper ion import | 56.44369748 | 2 | 0.0345 |
| GOTERM_BP_DIRECT | GO: 0002544~chronic inflammatory response | 43.90065359 | 2 | 0.0441 |
| GOTERM_BP_DIRECT | GO: 0007156~homophilic cell adhesion via plasma membrane | 5.001340283 | 4 | 0.0448 |
| | adhesion molecules | | | |
| KEGG_PATHWAY | hsa04350: TGF-beta signaling pathway | 8.471674877 | 3 | 0.0454 |
| KEGG_PATHWAY | hsa00450: Selenocompound metabolism | 27.90669371 | 2 | 0.0671 |
| GOTERM_BP_DIRECT | GO: 0001701~in utero embryonic development | 4.225731382 | 4 | 0.0673 |
| GOTERM_BP_DIRECT | GO: 0008284~positive regulation of cell proliferation | 2.543600101 | 6 | 0.0841 |
| GOTERM_BP_DIRECT | GO: 0010971~positive regulation of G2/M transition of mitotic | 21.9503268 | 2 | 0.0884 |
| | cell cycle | | | |

TABLE 6

| Category | Term | Fold Enrichment | Count | PValue |
|---|---|---|---|---|
| GOTERM_BP_DIRECT | GO: 0006334~nucleosome assembly | [symbol] | [symbol] | 4.79E−04 |
| GOTERM_BP_DIRECT | GO: 00072 [symbol] ~Ras protein signal transducion | [symbol] | [symbol] | [symbol] |
| KEGG_PATHWAY | hsa04810: Regulation of actin cytoskeleton | [symbol] | [symbol] | [symbol] |
| GOTERM_BP_DIRECT | GO: 0001525~angiogenesis | [symbol] | [symbol] | [symbol] |
| KEGG_PATHWAY | hsa05034: Alcoholism | [symbol] | 7 | [symbol] |
| GOTERM_BP_DIRECT | GO: 0017085~response to insecticide | [symbol] | 2 | 0.02309 |
| KEGG_PATHWAY | hsa05322: Systemic lupus erythematosus | [symbol] | [symbol] | [symbol] |
| GOTERM_OF_DIRECT | GO: 0032091~negative regulation of protein binding | [symbol] | 4 | [symbol] |
| KEGG_PATHWAY | hsa04070: [symbol] signaling system | [symbol] | [symbol] | 0.03253 |
| GOTERM_BP_DIRECT | GO: 0060978~angiogenesis involved in [symbol] y vascular morphogenesis | [symbol] | 2 | 0.03444 |
| GOTERM_BP_DIRECT | GO: 00 [symbol] 203~dGTP catabolic process | [symbol] | 2 | 0.03444 |
| GOTERM_BP_DIRECT | GO: 0045815~positive regulation of gene expression [symbol] | [symbol] | 4 | [symbol] |
| GOTERM_BP_DIRECT | GO: 0009611~response to wounding | [symbol] | 4 | [symbol] |
| GOTERM_BP_DIRECT | GO: 0043926~exonucleolytic nuclear-transcripted mRNA | [symbol] | 3 | 0.04437 |
| | catabolic process involved in deadenylation-dependent decay | | | |
| GOTERM_BP_DIRECT | GO: 0035404~histone-serine phosphorylation | [symbol] | 2 | 0.04555 |
| GOTERM_BP_DIRECT | GO: 0007 [symbol] 52~ [symbol] spindle organization | [symbol] | 3 | [symbol] |
| GOTERM_BP_DIRECT | GO: 0030855~epithelial cell differ [symbol] ation | [symbol] | 4 | 0.04791 |
| GOTERM_BP_DIRECT | GO: 00 [symbol] ~DNA replication-dependent nucleosome assembly | [symbol] | 3 | [symbol] |
| GOTERM_BP_DIRECT | GO: 00 [symbol] 1551~trige [symbol] inal ga [symbol] development | [symbol] | 2 | [symbol] |
| KEGG_PATHWAY | hsa04152: AMPK signaling pathway | [symbol] | [symbol] | [symbol] |
| GOTERM_BP_DIRECT | GO: 0 [symbol] 42 [symbol] ~wound healing | [symbol] | 4 | [symbol] |
| GOTERM_BP_DIRECT | GO: 0032958~i [symbol] phosphate biosynthetic process | [symbol] | 2 | [symbol] |
| GOTERM_BP_DIRECT | GO: 0071455~cellular response to hyper [symbol] | [symbol] | 2 | [symbol] |
| COTERM_BP_DIRECT | GO: 0051290~protein hetero [symbol] zation | [symbol] | 3 | [symbol] |
| GOTERM_BP_DIRECT | GO: 00 [symbol] 7 [symbol] ~cell-matrix adhesion | [symbol] | 4 | [symbol] |
| GOTERM_BP_DIRECT | GO: 005 [symbol] 597~response to meth [symbol] cury | [symbol] | 2 | [symbol] |
| GOTERM_BP_DIRECT | GO: 0 [symbol] 514~negative regulation of BMP signaling pathway | [symbol] | 3 | [symbol] |
| GOTERM_BP_DIRECT | GO: 00 [symbol] 627~regulation of vescile-mediatied transport | [symbol] | 2 | [symbol] |
| GOTERM_BP_DIRECT | GO: 0030903~ [symbol] development | [symbol] | 2 | [symbol] |

[symbol]

indicates data missing or illegible when filed

### Example 9: ITGB34 and PXN Maintain Genomic Stability and Mitochondrial Function

The Hallmark pathways represent specific well-defined biological processes and exhibit coherent expression. Of these 10 genes, G3B3P1, USP1, and VDAC1 were the top 3 downregulated genes constituting the MYC1 Hallmark pathway. To validate the RNAseq data, western blotting and qPCR experiments were performed. These results confirmed that knocking down either PXN or ITGB34, or both, resulted in the decreased expression of all 3 genes at both protein level (FIG. 7F) as well as RNA level (FIG. 70). Furthermore, the expression of these genes in lung TOGA data set was also analyzed to determine the expression of USP1 and VDAC1 in tumor and normal samples (FIG. 7G). Next, whether knocking down these genes would recapitulate the phenotype of the ITGB4/PXN double knockdown was investigated. The siRNA sequences are provided below in Table 7:

TABLE 7
siRNA Sequence

| Target | Locus | | | |
|---|---|---|---|---|
| Gene | ID | RefSeq | Construct | Sequence (5′-3′) |
| PXN | 5829 | NM_001080855.2, | — | GCUUCGCUGUCGGAUUUCATT (SEQ ID NO: 6) |
| | | NM_001243756.1, | | |
| | | NM_002859.3, | | |
| | | NM_025157.4 | | |
| ITGB4 | 3691 | NM_000213, | A | CAGUUCUGCGAGUAUGACAACUUCC (SEQ ID NO: 7) |
| | | NM_001005619, | B | CGAGAAGCUUCACACCUAUUUCCCT (SEQ ID NO: 8) |
| | | NM_001005731, | C* | GUCAGUUCUGCGAGUAUGACAACTT (SEQ ID NO: 9) |
| | | NM_001321123 | | |
| USP1 | 7398 | NM_001017415, | A | AGCUACAAGUGAUACAUUAGAGAGT (SEQ ID NO: 10) |
| | | NM_001017416, | B* | GGAGCACAAAGCCAACUAACGARCA (SEQ ID NO: 11) |
| | | NM_003368 | C | GGCAAGUUAUGAAUUGAUAUGCAGT (SEQ ID NO: 12) |
| VDAC1 | 7416 | NM_003374, | A* | ACAACACUCAGAAUCUAAAUUGGAC (SEQ ID NO: 13) |
| | | NR_036624, | B | GGAAUUUCAAGCAUAAAUGAAUACT (SEQ ID NO: 14) |
| | | NR_036625 | C | GCACCAGAGUAUGAAAUAGCUUCCA (SEQ ID NO: 15) |
| ITGA7 | 3679 | NM_001144996, | A | AGUGAAGUCCUCCAUAAAGAACUTG (SEQ ID NO: 16) |
| | | NM_001144997, | B | GUAUGAGGUCACGGUUUCCAACCAA (SEQ ID NO: 17) |
| | | NM_002206 | C | CUUAGUUUGCUGCCAUCAGUCUAGT (SEQ ID NO: 18) |
| KIF14 | 9928 | NM_001305792, | A | GCACUGACAAGAAAUGUUAUAAAGA (SEQ ID NO: 19) |
| | | NM_014875 | B | CACGAUCACAGAAUAACAAGUCGAA (SEQ ID NO: 20) |
| | | | C | GGCAUAACUAGUUGAAAUUGGCCAT (SEQ ID NO: 21) |
| G3BP1 | 10146 | NM_005754, | A | GUGCGAGAACAACGAAUAAAUAUTC (SEQ ID NO: 22) |
| | | NM_198395 | B | CCCGUAAGAAGGAAUGUUACUUUAA (SEQ ID NO: 23) |
| | | | C | ACCACCUCAUGUUGUUAAAGUACCA (SEQ ID NO: 24) |

*Denotes optimal siRNA construct used for downstream experiments

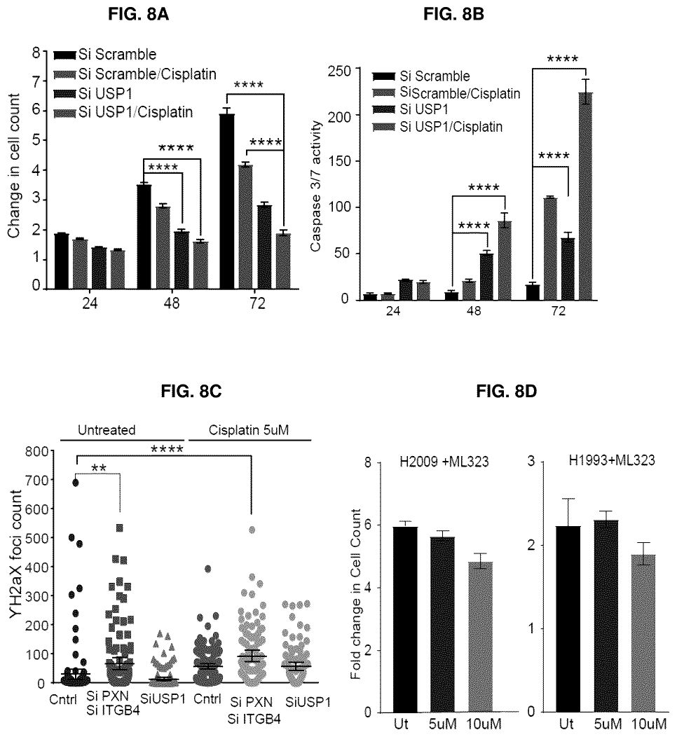

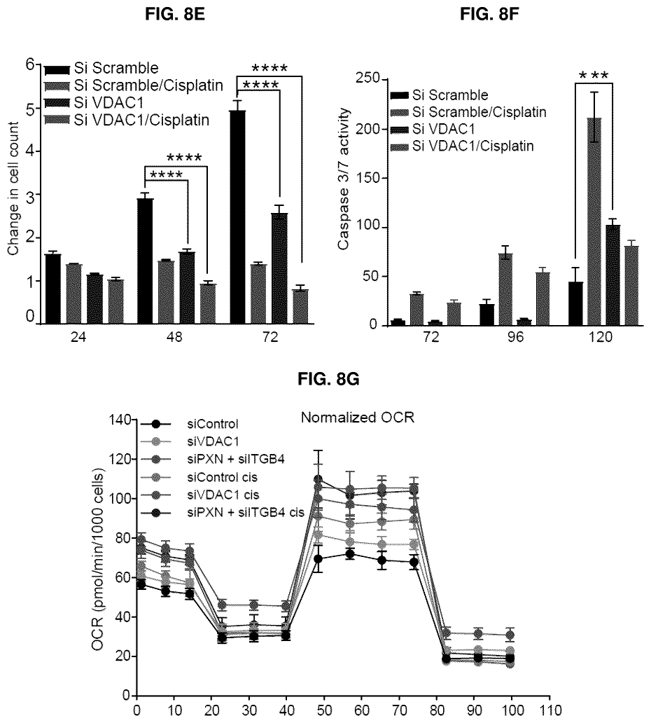

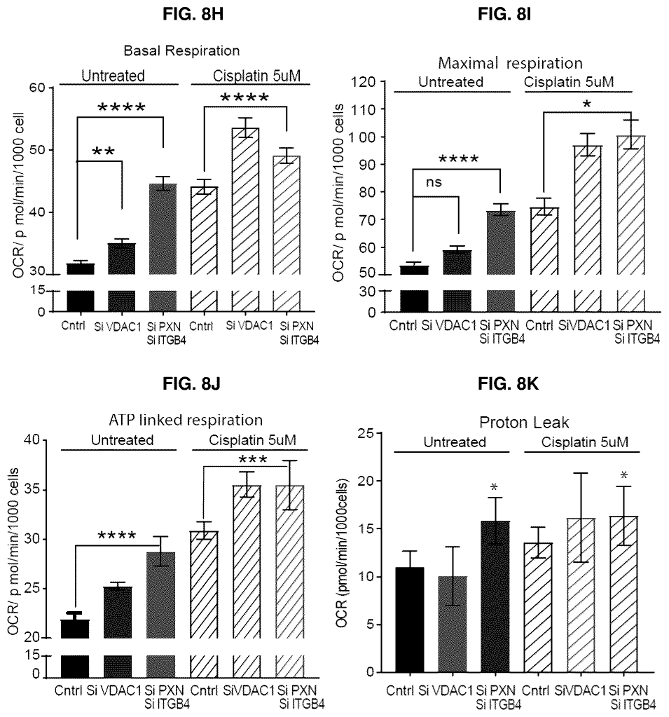

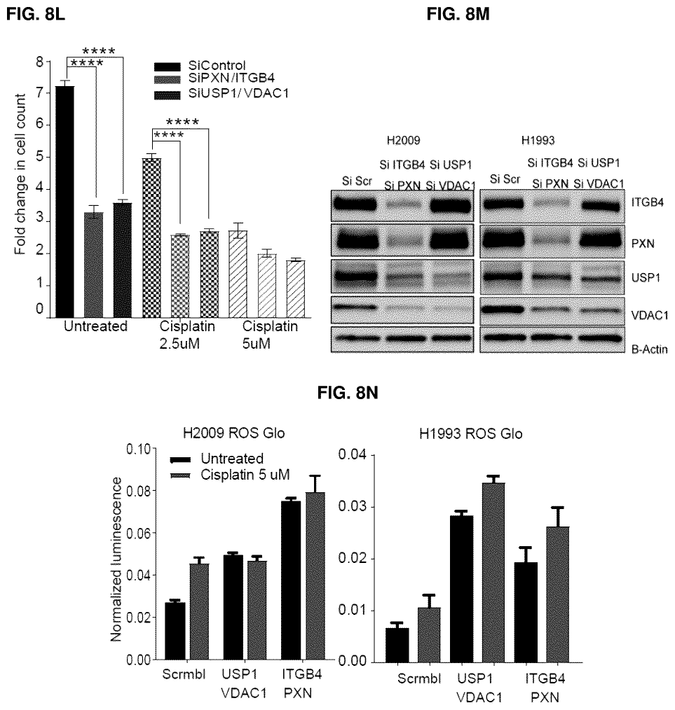

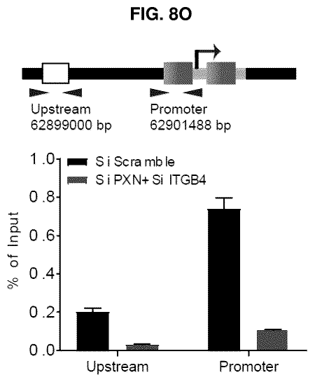

USP1 knockdown inhibited cell proliferation >2-fold by 72 hours of seeding and simultaneously increased caspase activity. Furthermore, USP1 knockdown in H2009 cells also sensitized them to a lower (2 μM) dose of cisplatin (FIGS. 8A and 81B). The sensitization of H2009 and H1993 cells to the USP1-selective inhibitor ML323 was also tested (Qin et al). The cells were treated with ML323 for 3 days with 5 μM and 10 μM but no significant change in the cell proliferation rates was observed (FIG. 8D). These data indicate that phenotype of USP1 knockdown was due to decrease in USP1 level whereas the phenotype of drug was due to disruption of USP1-UAF1 complex indicating the expression of USP1 is crucial for lung cancer cell survival. Knocking down VDAC1 led to a decrease in cell proliferation by 50% by 72 hours. However, silencing VDAC1 expression did not contribute to apoptosis induction at early time point even in the presence of cisplatin although, at a later (120 hours) time point, an increase in apoptosis by 2-fold was observed (FIGS. 8E and 8F). The possibility of decreased expression of ITGB4 and PXN by knocking down USP1 or VDAC1 was checked, but no change was observed.

Taken together, these data suggest that USP1 and VDAC1 are downstream of PXN and ITGB4 and thus, knocking down USP1 and VDAC1 should recapitulate the phenotype of PXN and ITGB4. Therefore, H2009 cell line was transfected with 10 nM of siRNA against ITGB4 and PXN or siRNA against USP1 and VDAC1 and changes in proliferation rates were compared. Cell proliferation was 50% reduced by knocking down either of the combination. Addition of cisplatin to either combination had an additive effect. Interestingly, there was significant difference between either gene combinations (FIG. 8L). This suggests that USP1 and VDAC1 plays a critical role in the tumor cell proliferation and thus exploring their functionality will provide more insight of their role in lung cancer development.

VDAC1 is an ion channel pump located in the mitochondrial and plasma membranes. To discern the role of VDAC1 in mitochondrial function, VDAC1 as well as PXN and ITGB4 was knocked down in H2009 cells and mitochondrial respiration in absence and presence of cisplatin was analyzed using the Seahorse XF Analyzer (Agilent, Santa Clara, CA). The raw data was normalized to the live cell count. Knocking down VDAC1 or ITGB4/PXN changed the oxygen consumption rate (OCR) of cells which is measured by the basal as well as maximal mitochondrial OCR compared to the control, and these rates were further increased in presence of cisplatin (FIGS. 8G, 8H, and 8I).

Oxidative phosphorylation is an efficient but slow process of ATP production in comparison to glycolytic pathway and is more preferably used by tumor cells. Pyruvate generated during glycolysis is oxidized by mitochondria for oxidative phosphorylation leading to mitochondrial ATP production which is required for ATP-linked respiration (Ajit et al.). Treating cells with Oligomycin which can inhibit the electron transport chain and block mitochondrial ATP generation, can inhibit ATP-linked respiration. On one hand, increase in ATP-linked respiration is indicative of the availability of more substrates like pyruvate to drive oxidative phosphorylation. On the other hand, it also suggests an increase in ATP demand of the cell. Therefore, ATP-linked respiration was calculated and an increase in the linked respiration was observed in double knockdown cells which increased further upon addition of cisplatin. Control (scramble siRNA treated) cells, in the presence of cisplatin showed increased ATP-linked respiration which is indicative of a stress-induced increase in ATP demand. The same increase in ATP demand was also seen in these cells upon VDAC1 knockdown or double knockdown (FIG. 8J).

Mitochondria maintain a proton motive potential for ATP generation under ideal conditions. A proton leak can affect the membrane potential and consequently, a decrease in ATP production. To maintain the proton motive force intact, mitochondria increase their respiration and oxygen consumption rate as measured by increase in basal and maximal respiration. An increase was observed in the proton leak for ITGB4 and PXN double knockdown which may explain their increase in respiration to compensate for the loss in membrane potential (FIG. 8K). Further, respiratory capacity is measured by considering the ratio of maximal respiration compared to basal respiration which does not show much difference across samples.

Increased mitochondrial respiration is also known to cause an increase in the generation of reactive oxygen species (ROS). Therefore, the production of ROS was tested using ROS glow kit form Promega in 2 cisplatin-resistant cell lines by knocking down either USP1/VDAC1 or ITGB4/PXN. Increase in mitochondrial oxygen consumption rate (OCR) is associated with increase in mitochondrial ROS post irradiation leading to cell death (Tohru Yamamori et al). An increase in generation of ROS was observed after knocking down ITGB4/PXN or USP1/VDAC1 which was higher compared to cells treated with cisplatin which may lead to increase in ROS induced DNA damage and apoptosis (FIG. 8N).

Furthermore, USP1 as a deubiquitinase is known to increase DNA repair activity in cisplatin-treated cells (Iraia et al). Therefore, changes in the γH2AX foci were measured as a measure of DNA damage in the double knockdown or USP1 knockdown cells. It was observed that double knockdown cells had significantly higher γH2AX foci counts compared to control or USP1 knockdown. Addition of cisplatin in conjunction with USP1 knockdown further increased the extent of DNA damage (FIG. 8C).

Finally, since double knockdown silenced USP1 expression alluding to possible transcriptional regulation, the upstream sequence of USP1 promoter region was analyzed using genome browser and two potential histone H3K27 acetylation sites were identified. To determine the functional significance of these sites, both PXN and ITGB4 were knocked down and after 72 hours, chromatin immunoprecipitation was performed using a histone H3K27 antibody. In the double knockdown samples, reduced histone H3K27 acetylation at the promoter was observed suggesting that USP1 expression is induced by hyper acetylation. Of note, the data also suggest that ITGB4 and PXN involvement is not limited to migration and invasion; they may also be involved in controlling the epigenetic land scape of lung cancer. However, additional studies are needed at global level and with other histone acetylation forms (FIG. 8O).

### Example 10: Biochemical Data Demonstrate Direct Interaction Between PXN and ITGB4

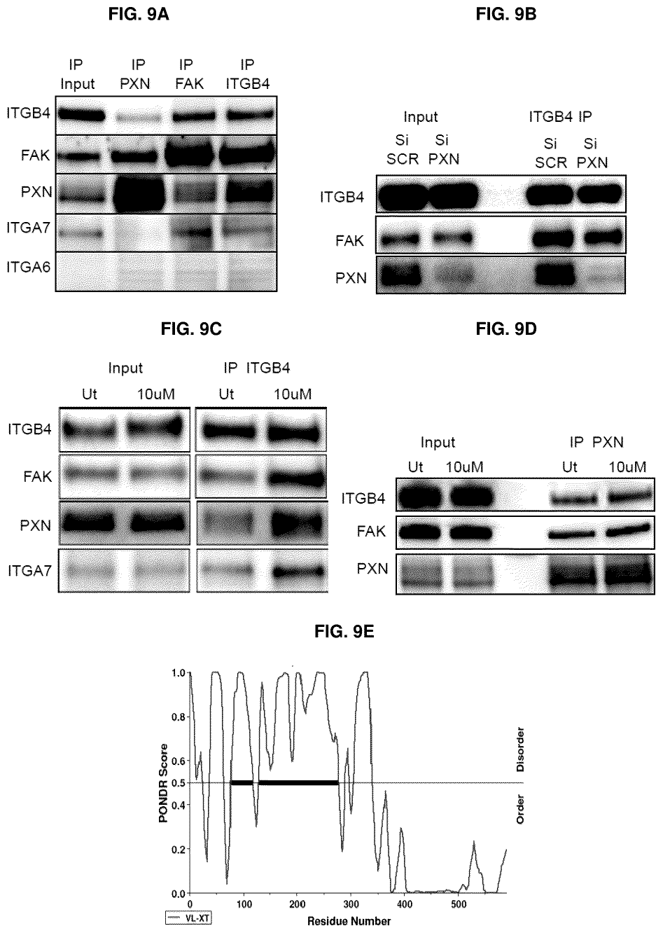

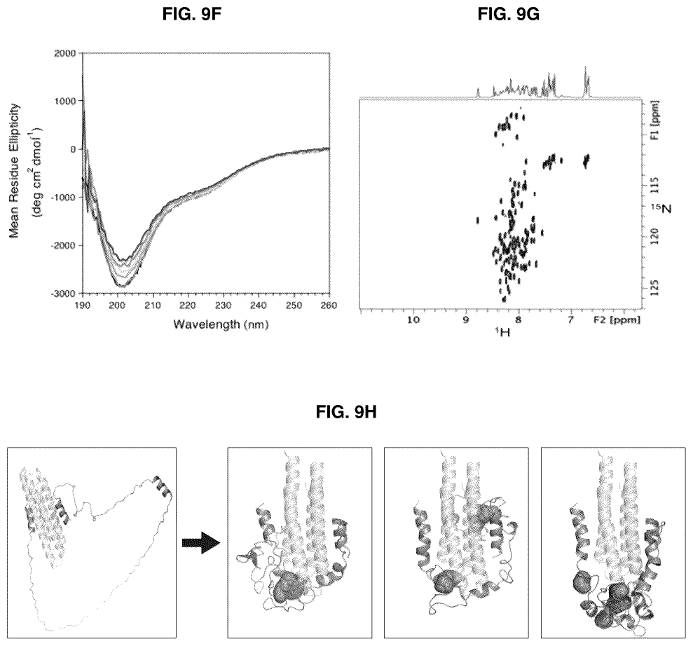

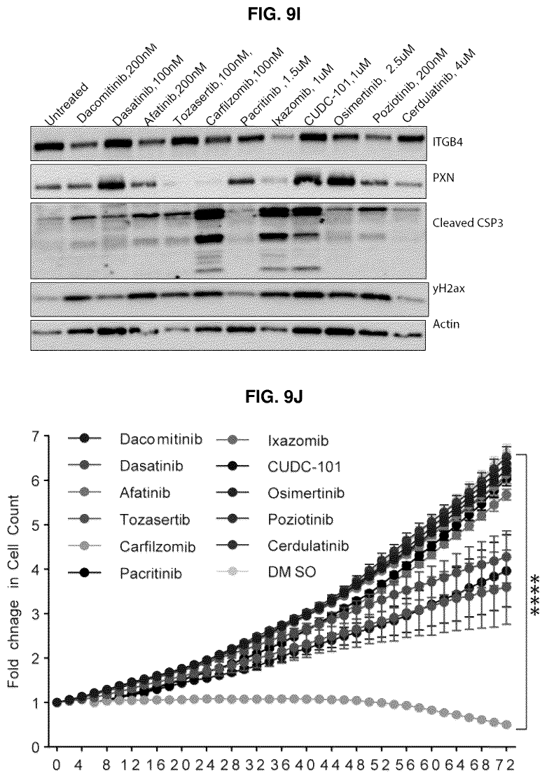

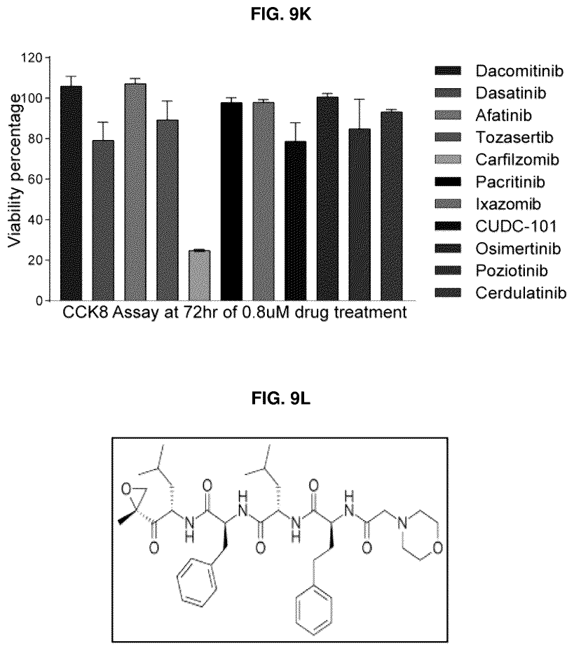

From the foregoing, it follows that PXN and ITGB4 interact with each other and also with FAK and that this interaction is important in cisplatin resistance. To determine these interactions, co-immunoprecipitation experiments were performed with an ITGB4 antibody as well as with FAK or PXN antibodies in separate experiments. Immunoblotting with ITGA6 antibody served as a positive control as it is known to interact with ITGB4 (Aydin et al). Surprisingly, the ITGA6 protein was not detected in H2009 and H1993 cell lines. However, the pulldown showed the presence of ITGA7 (FIG. 9A). Furthermore, the immunoprecipitation data indicated a stronger interaction between FAK and ITGB4 compared to PXN and ITGB4, suggesting that FAK interaction with ITGB4 is PXN-dependent or vice versa. To test this possibility, PXN was knocked down and immunoprecipitation was repeated with the ITGB4 antibody. This experiment demonstrated that indeed, FAK interacts with ITGB4 irrespective of PXN (FIG. 9B). Next, immunoprecipitations were performed using lysates from cells treated with cisplatin for 48 hours to determine if cisplatin induced any change in the interaction of ITGB4, FAK, and PXN (FIGS. 9C and 9D). By immunoprecipitating ITGB4 as well as PXN, no change in the interactions was observed among FAK, PXN, and ITGB4 in presence of cisplatin, which again indicated the involvement the FA complex in imparting cisplatin resistance. Considered together, the co-immunoprecipitation data suggested that disrupting interaction between PXN, ITGB4, and FAK in cisplatin-resistant cells could make them more susceptible to cisplatin.

### Example 11: The N-Terminal Region of PXN is Intrinsically Disordered

Since PXN, especially the LD1-LD5 domain, is implicated in interacting with multiple proteins in the FA complex, it is suspected that this region may be intrinsically disordered. Bioinformatics analysis using the PONDR prediction algorithm showed that indeed, the N-terminal half of the molecule, especially the regions connecting the LD domains, is predicted to be significantly disordered while the C-terminal half comprising the LIM domains is predicted as highly ordered (FIG. 9E). To experimentally verify the prediction, circular dichroism (CD) and NMR spectroscopy were employed. CD spectra were recorded as a function of temperature from 10° C. to 60° C. These spectra showed that the N-terminal region of PXN polypeptide chain lacks significant α-helical or β-strand secondary structural elements over this temperature range as evidenced by low ellipticity values in the 215-230 nm region. However, a weak ellipticity minimum at ˜222 nm was observed, suggesting some helical character which could likely correspond to the short helical regions (5-7 amino acids) between the large disordered regions (FIG. 9F). NMR was used to corroborate the CD data. A two-dimensional 1H-15N HSQC spectrum of the LD2-LD4 region (FIG. 9G) confirmed that this domain is indeed disordered. Taken together, the biochemical, computational, and biophysical experiments not only provide good evidence that PXN is an IDP and therefore can interact with multiple partners, but also suggest that the interactions between PXN, ITGB4, and FAK contribute to cisplatin resistance in lung cancer. Consistently, the data demonstrating a strong synergistic effect of the double knockdown underscored the importance of the PXN/ITGB4 complex in drug resistance in these cells.

### Example 12: FDA-Approved Drugs Alleviate Cisplatin Resistance in LUAD

A virtual screen of a library of 1440 FDA-approved drugs using binding pockets in the N-terminal domain of PXN (FIG. 9H) identified several putative hits. The top 40 hits were then rescreened employing a cell-based assay to determine their efficacy using H358 cells. The H358 cells were used because they have high expression of ITGB4/PXN and exhibit intermediate sensitivity to cisplatin. Cells were treated with various concentrations of the drugs (0.1 μM to 10 μM) for 72 hours and the IC50 values were determined (Table 8).

TABLE 8

| Drug | IC50 (uM) |
|---|---|
| Carfilzomib | 0.0008 |
| Tozasertib | 0.0009 |
| Dasatinib | 0.001 |
| Afatinib | 0.009 |
| Dacomitinib | 0.06 |
| Poziotinib | 0.06 |
| Pacritinib | 0.1714 |
| Ixazomib | 0.303 |
| Osimertinib | 0.358 |
| CUDC-101 | 0.501 |
| Cerdulatinib | 1.54 |

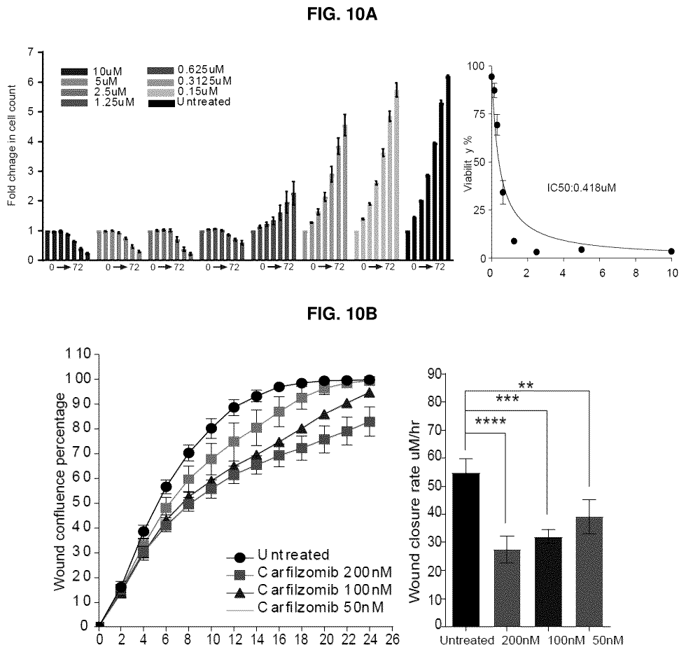

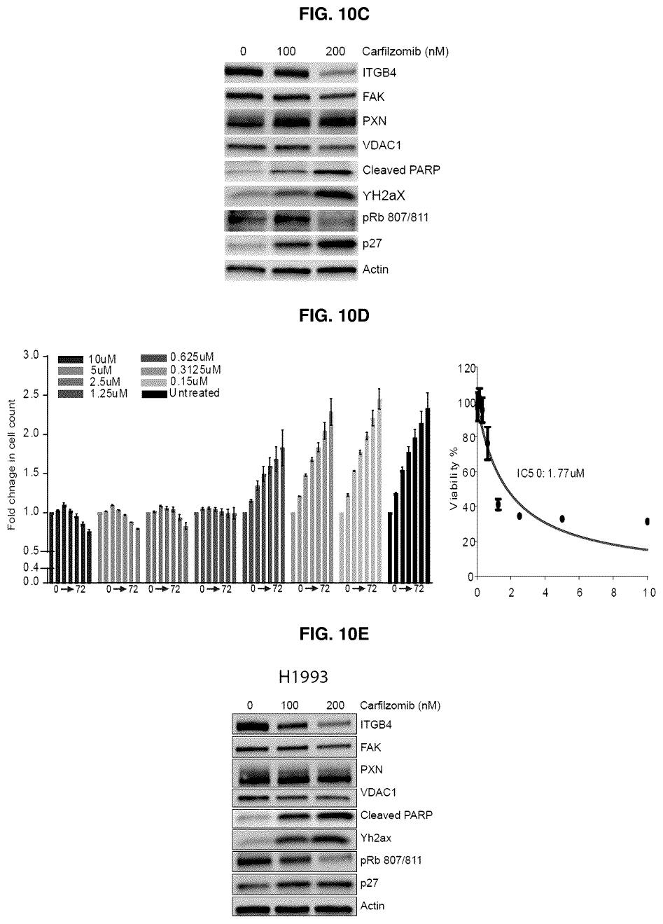

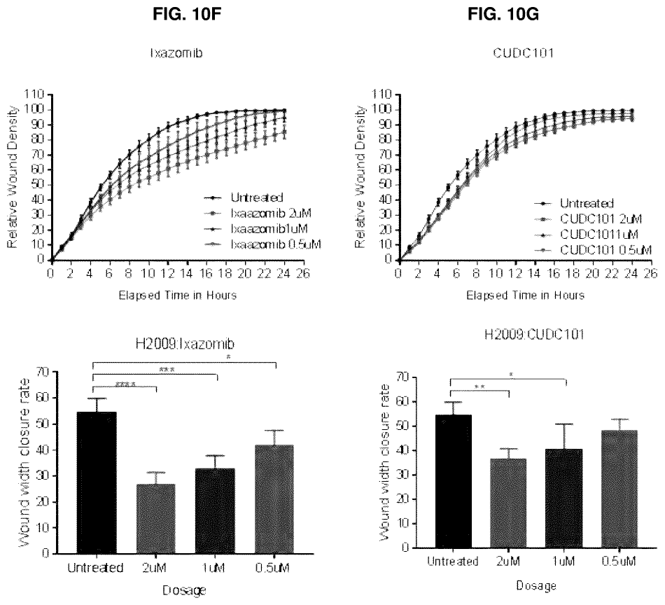

Eleven potential compounds were identified, which showed a significant effect with IC50s ranging from 0.8 nM to 1.54 μM. Then a sublethal dose was used to determine the effect of these compounds on ITGB4 and PXN expression and caspase activity (FIG. 9I). Three of the 11 compounds carfilzomib, ixazomib, and CUDC-101 induced apoptosis and caused DNA damage measured by γH2AX accumulation and decrease in ITGB4 expression. These eleven compounds were further screened in the cisplatin-resistant cell line H2009. After treating cells with 80 nM dose of each compound for 72 hours, carfilzomib (FIG. 9L) had the greatest inhibitory effect in cell proliferation (FIG. 10D) and reduction in cell viability (FIG. 10E) with an IC50 of 0.418 μM (FIG. 10A). Immunoblotting of H2009 cells treated with a sublethal dose of the eleven compounds also confirmed that only carfilzomib reduced ITGB4 and PXN expression (FIG. 10F). Furthermore, significant inhibition of wound healing and reduction in wound closure rate in H2009 cells was confirmed when treated with sublethal doses of 200, 100, or 50 nM of carfilzomib (FIG. 10B). Ixazomib and CUDC-101 on the other hand, were less effective than carfilzomib in inhibiting wound healing. Immunoblotting experiments using cell extracts from H2009 cells treated with a sublethal dose of carfilzomib showed a reduction in ITGB4 expression but an increase in the levels of p27, γH2AX, and cleaved PARP (FIG. 10C). Further, carfilzomib was tested in another cisplatin-resistant cell line H1993. Carfilzomib was also effective in this cell line with an IC50 of 1.7 μM (FIG. 8E). Finally immunoblotting using lysates from H1993 cells treated with 50-200 nM carfilzomib showed reduction in ITGB4 expression and increase in cleaved PARP and γH2AX, indicating cell death.

### Example 13: Carfilzomib Inhibited Patient Derived Organoids Better than Cisplatin

The experiments described above were done using 2D monolayer cultures. H2009 spheroids were employed to discern the effect of the drug in 3D cultures. The spheroids were treated with different doses (150-600 nM) for 72 hours and spheroid integrity was analyzed by measuring the red and green intensities using confocal microscopy. Consistent with the 2D data, a significant increase in caspase activity and decrease in cell proliferation was observed (FIGS. 11A-11C). The confocal images taken 72 hours post-treatment showed intact spheroids in the untreated condition but disintegrated spheroids with high green fluorescence in the presence of carfilzomib.

Next, drug efficacy in patient tumor tissue-derived organoids was determined. Approximately 5000 cells/sample from 2 surgical samples were seeded in an ultra-low attachment plate and incubated at 37° C. Once organoids formed, carfilzomib and caspase assay dye were added, and the organoids were observed in real time using the IncuCyte Live Cell Analysis System. In one of the patient-derived organoids, cisplatin treatment alone decreased spheroid area and increased apoptosis as measured by green intensity (FIG. 11D). Furthermore, treating with carfilzomib also decreased spheroid growth and increased apoptosis. Indeed, by 72 hours carfilzomib at a concentration of 2.5 μM induced 5-fold increase in caspase activity compared to cisplatin alone but there was not much change in the spheroid area (FIGS. 11E-11F). Thus, no additive effect was apparent in this patient sample. However, in the second case, cisplatin did not induce any caspase activity but carfilzomib by itself induced ˜50-fold increase in caspase activity which was further enhanced when the two drugs were combined (FIGS. 11G-11I). The efficacy of ITGB4 antibody in disrupting organoid growth and inducing apoptosis was also tested. After 6 days of treatment, a significant decrease in organoid area was observed suggesting that ITGB4 antibody treatment significantly affects patient-derived organoid growth compared to untreated or 2.5 μM cisplatin-treated but could not disrupt the organoid or induce cytotoxicity. Considered together, these data indicated that in patients who are resistant to cisplatin, sensitivity can be enhanced by using carfilzomib.

Next, the organoids were treated with cisplatin alone or carfilzomib at 2.5 mM and 5 mM for 48 hours and the samples were processed for immunoblotting. A decrease in the expression of ITGB4, USP1, PXN and VDAC1 was observed, which were found to be associated with cisplatin resistance in the present study (FIG. 11J). In addition, decrease in CD133 expression was observed, which is a marker for cancer initiating cells which are thought to be responsible for acquired drug resistance (Bertolini G et al). Thus, carfilzomib treatment can sensitize the tumor cells as well as the cancer initiating cells, suggesting a potential drug for cisplatin refractory lung cancer cases.

### Example 14: Mathematical Modeling Indicates Bistability

Analysis of RNAseq data revealed that several microRNAs were also differentially regulated in response to single or double knockdown (Table 9).

TABLE 9

| | | |
|---|---|---|
| | Predicted | |
| Activation Score | Activation | P-value |

ITGB4 Knockdown

| | | | |
|---|---|---|---|
| miR-223 | 2.236 | Activated | 0.0985 |
| miR-210 | 2.449 | Activated | 0.00772 |
| miR-124-3p | 2.077 | Activated | 0.00352 |
| miR-1-3p | 3.128 | Activated | 0.0000133 |

PXN KD

| | | | |
|---|---|---|---|
| miR-199a-5p | −2.43 | Inhibited | 0.000731 |

PXN/ITGB4 Knockdown

| | | | |
|---|---|---|---|
| miR-291a-3p | 2.562 | Activated | 1.25E−08 |
| miR-124-3p | 2.292 | Activated | 0.0000371 |
| miR-30c-5P | 2.345 | Activated | 0.0081 |
| miR-1-3p | 2.543 | Activated | 0.00296 |
| miR-199a-5p | −2.135 | Inhibited | 0.0015 |

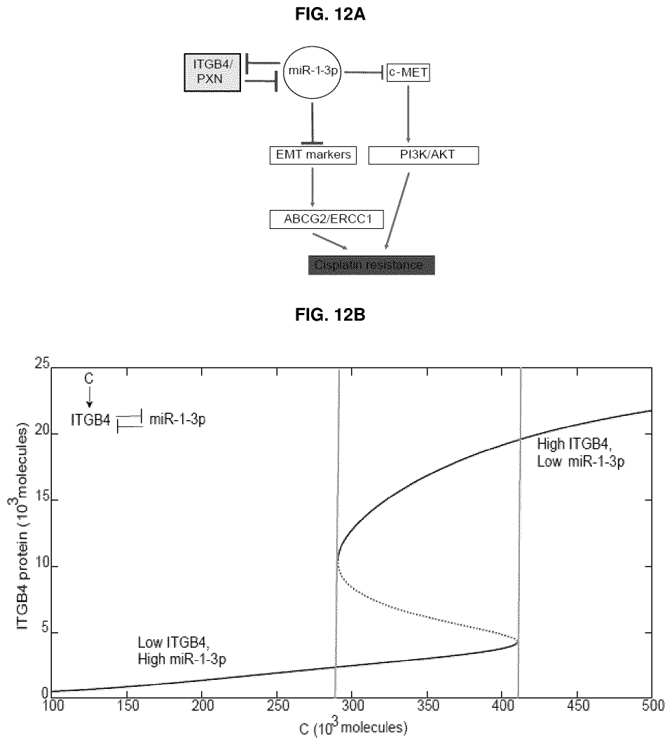

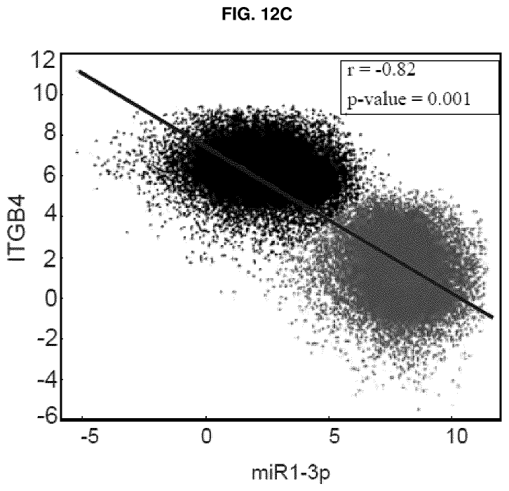

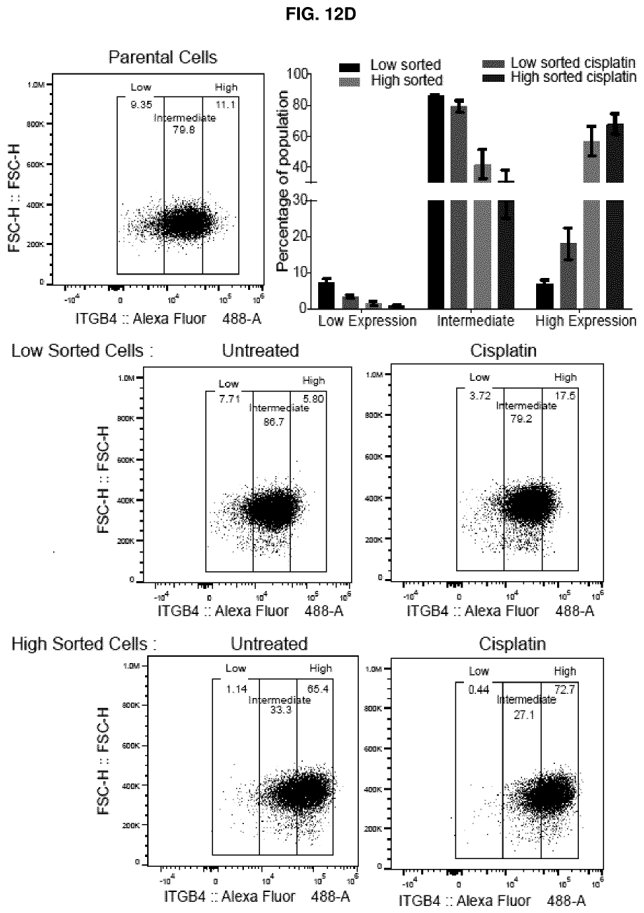

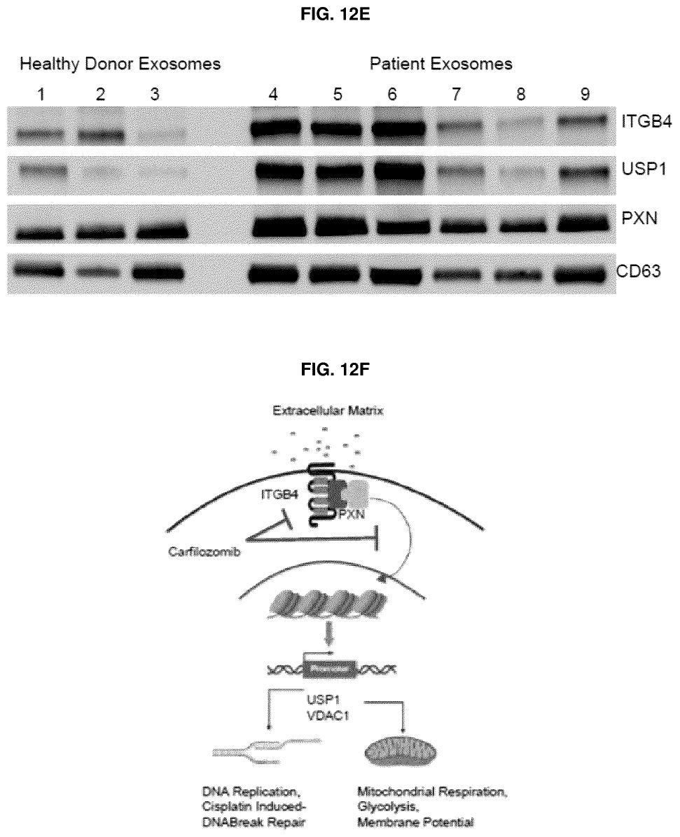

In particular, miR-1-3p was upregulated both in ITGB4 single knockdown as well as in the ITGB4/PXN double knockdown by 3.1-fold and 2.54-fold, respectively. Furthermore, it was reported that overexpressing ITGB4 downregulates miR-1-3p (Gerson et al, 2012). Together, these observations suggested that there exists a double negative feedback loop between ITGB4 and miR-1-3p. Therefore, integrating these observations, a mathematical model was developed to simulate the dynamics of ITGB4/miR-1-3p feedback loop (FIG. 12A). This model showed the existence of two stable states (phenotypes): one (sensitive) state represented by (low ITGB4, high miR-1-3p) and the other (resistant) state represented by (high ITGB4, low miR-1-3p) (FIG. 12B). Moreover, the model suggested it is possible for cells to switch their state under the influence of biological noise or stochasticity; thus, a sensitive cell can behave as resistant and vice-versa.

Next, to investigate whether this co-existence of two states is a robust feature that can be expected from the given network topology, the algorithm RACIPE (Random Circuit Perturbation) was implemented, which generates an ensemble of mathematical models with varying parameter sets (Huang et al, 2017). The results from this ensemble (n=100,000) were then plotted together to identify robust dynamical features of the underlying regulatory network; each mathematical model can represent one cell, and this ensemble represents a cell population with varying levels of genetic/phenotypic heterogeneity. The feedback loop was constructed based on data reported in the manuscript (ITGB4 inhibits miR-1-3p) and publicly available data (miR-1-3p inhibits ITGB4)—www.genecards.org/cgi-bin/carddisp.pl?gene=ITGB4. RACIPE results for the ITGB4/miR-1-3p feedback loop showed that both ITGB4 and miR-1-3p exhibit bimodality, i.e., two subpopulations (FIGS. 12B and 12C). These two subpopulations were also distinctly observed in a scatter plot of ITGB4/miR-1-3p showing negative correlation (r=−0.82, p<0.001), reinforcing the previous results indicating the existence of two states—high ITGB4, low miR-1-3p and low ITGB4, high miR-1-3p. All in all, these results indicate that ITGB4/miR-1-3p feedback loop enables phenotypic plasticity.

To test the predictions made by the model, H2009 cells were treated with cisplatin for 4 days. On day 5, they were subjected to FACS analysis using an ITGB4 antibody. A 15% decrease in ITGB4 low-expressing cells was observed compared to untreated cells, suggesting that the H2009 cells are inherently a mix population with variable expression of ITGB4. In presence of stress, the cells either increased the expression of ITGB4 or the cells having high ITGB4 get selected (FIG. 13B). Additionally, 10% of median low ITGB4 expressing cells (Low sorted) and 10% of media high ITGB4 subpopulations were gated and purified by FACS sorting and they were cultured separately. The sorted cells were seeded in 6 independent wells and allowed to proliferate. After 2 days, 3 of 6 wells were treated with a sub-lethal dose of cisplatin for 2 days. The cells were then counted, and an equal number of cells were stained with ITGB4 antibody and PI and analyzed by FACS sorting. Interestingly, the ITGB4-low cells recreated ITGB4-intermediate and ITGB4-high population while ITGB4-high sorted cells kept proliferating. Finally, these cells were treated with cisplatin and a shift in population for the low and intermediate gated cells towards the higher gate was observed, suggesting that (cisplatin) stress favored the population with high expression of ITGB4 (FIG. 12D).

### Example 15: Exosomal ITGB4 and PXN as Biomarkers for Predicting Cisplatin Response

Since ITGB4 and PXN are upregulated in cisplatin-resistant cells, a minimally invasive method could be developed to determine the expression of ITGB4 and PXN in LUAD patients. To this end, 500 μl of serum from healthy donors and LUAD patients was used and exosomes were isolated by ultracentrifugation as described in Example 1. Exosomes were lysed and analyzed for ITGB4 and PXN expression by western blotting. CD63 served as an exosomal marker protein. Indeed, of six patients that were tested, three patients had higher expression of ITGB4 and PXN. Interestingly, USP1 which was identified as being regulated by ITGB4 showed a similar pattern corresponding to ITGB4 (FIG. 12E). These data suggest that ITGB4 and PXN represent attractive predictive biomarkers for cisplatin resistance.

### REFERENCES

Acar, M., Becskei, A. & van Oudenaarden, A. Enhancement of cellular memory by reducing stochastic transitions. Nature 2005 435, 228-232.
Alberts B, Johnson A D, Lewis J, Morgan D, Raff M, Roberts K, Walter P. Molecular Biology of the Cell 6th Edition 2014, Garland Science Publishers.
Alvarez-Arenas A, Podolski-Renic A, Belmonte-Beitia J, Pesic M, Calvo G F. Interplay of Darwinian Selection, Lamarckian Induction and Microvesicle Transfer on Drug Resistance in Cancer. Sci Rep. 2019 9(1):9332.
Ambadipudi S, Zweckstetter M. Targeting intrinsically disordered proteins in rational drug discovery. Expert Opin Drug Discov. 2016 11(1):65-77.
Ajit S. Divakaruni, Alexander Paradyse, David A. Ferrick, Anne N. Murphy, Martin Jastroch. Analysis and interpretation of microplate-based oxygen consumption and pH data. Methods Enzymol. 2014; 547: 309-354.
Balázsi, G., van Oudenaarden, A. & Collins, J. J. Cellular decision making and biological noise: from microbes to mammals. Cell 2011 144, 910-925.
Barabasi A L. Scale-free networks: a decade and beyond. Science. 2009 325(5939):412-3.
Barabasi A L, Albert R. Emergence of scaling in random networks. Science. 1999 286(5439):509-12.
Berg T. Small-molecule modulators of c-Myc/Max and Max/Max interactions. Curr Top Microbiol Immunol. 2011; 348:139-149.
Bertolini G, Roz L, Perego P, Tortoreto M, Fontanella E, Gatti L et al. Highly tumorigenic lung cancer CD133+ cells display stemlike features and are spared by cisplatin treatment. Proc Natl Acad Sci USA 2009; 106: 16281-16286.
Bray F, Ferlay J, Soerjomataram I, Siegel R L, Torre L A, Jemal A. Global cancer statistics 2018: GLOBOCAN estimates of incidence and mortality worldwide for 36 cancers in 185 countries. C A Cancer J Clin. 2018 68(6):394-424.
Charlebois, D. A., Abdennur, N. & Kaern, M. Gene expression noise facilitates adaptation and drug resistance independently of mutation. Phys. Rev. Lett. 2011 107.
Chen C S, Alonso J L, Ostuni E, Whitesides G M, Ingber D E. Cell shape provides global control of focal adhesion assembly. Biochem Biophys Res Commun. 2003 307(2):355-61.
Deben C, Deschoolmeester V, De Waele J, Jacobs J, Van den Bossche J, Wouters A, Peeters M, Rolfo C, Smits E, Lardon F, Pauwels P. Hypoxia-Induced Cisplatin Resistance in Non-Small Cell Lung Cancer Cells Is Mediated by HIF-1α and Mutant p53 and Can Be Overcome by Induction of Oxidative Stress. Cancers (Basel). 2018 10(4). pii: E126.
Dong J M, Lau L S, Ng Y W, Lim L, Manser E. Paxillin nuclear-cytoplasmic localization is regulated by phosphorylation of the LD4 motif: evidence that nuclear paxillin promotes cell proliferation. Biochem J. 2009 418(1):173-84.
Engl C. Noise in bacterial gene expression. Biochem Soc Trans. 2019 47(1):209-217
Farquhar K S, Charlebois D A, Szenk M, Cohen J, Nevozhay D, Balázsi G. Role of network-mediated stochasticity in mammalian drug resistance. Nat Commun. 2019 10(1):2766.
Fennell D A, Summers Y, Cadranel J, Benepal T, Christoph D C, Lal R, Das M, Maxwell F, Visseren-Grul C, Ferry D. Cisplatin in the modern era: The backbone of first-line chemotherapy for non-small cell lung cancer. Cancer Treat Rev. 2016 44:42-50.
Galluzzi L, Senovilla L, Vitale I, Michels J, Martins I, Kepp O, Castedo M, Kroemer G. Molecular mechanisms of cisplatin resistance. Oncogene. 2012 31(15):1869-83.
Galluzzi L, Vitale I, Michels J, Brenner C Szabadkai G, Harel-Bellan A, Castedo M, Kroemer G. Systems biology of cisplatin resistance: past, present and future. Cell Death Dis. 2014 5:e1257.
Gerlinger M, Swanton C. How darwinian models inform therapeutic failure initiated by clonal heterogeneity in cancer medicine. Br. J. Cancer. 2010 103:1139-1143.
Gerson K D, Shearstone J R, Maddula V S, Seligmann B E, Mercurio A M. Integrin β4 regulates SPARC protein to promote invasion. J Biol Chem. 2012 287(13):9835-44.
Giancotti F G, Ruoslahti E. Integrin signaling. Science. 1999 285(5430):1028-32.
Hoshino A, Costa-Silva B, Shen T L, Rodrigues G, Hashimoto A, Tesic Mark M, Molina H, Kohsaka S, Di Giannatale A, Ceder S, Singh S, Williams C, Soplop N, Uryu K, Pharmer L, King T, Bojmar L, Davies A E, Ararso Y, Zhang T, Zhang H, Hernandez J, Weiss J M, Dumont-Cole V D, Kramer K, Wexler L H, Narendran A, Schwartz G K, Healey J H, Sandstrom P, Labori K J, Kure E H, Grandgenett P M, Hollingsworth M A, de Sousa M, Kaur S, Jain M, Mallya K, Batra S K, Jarnagin W R, Brady M S, Fodstad O, Muller V, Pantel K, Minn A J, Bissell M J, Garcia B A, Kang Y, Rajasekhar V K, Ghajar C M, Matei I, Peinado H, Bromberg J, Lyden D. Tumour exosome integrins determine organotropic metastasis. Nature. 2015 527(7578):329-35.
Herbst R S, Morgensztern D, Boshoff C. The biology and management of non-small cell lung cancer. Nature. 2018 553(7689):446-454.
Hu W, Jin P, Liu W. Periostin Contributes to Cisplatin Resistance in Human Non-Small Cell Lung Cancer A549 Cells via Activation of Stat3 and Akt and Upregulation of Survivin. Cell Physiol Biochem. 2016; 38(3):1199-208.
Hu G, Wu Z, Uversky V N, Kurgan L. Functional Analysis of Human Hub Proteins and Their Interactors Involved in the Intrinsic Disorder-Enriched Interactions. Int J Mol Sci. 2017 18(12). pii: E2761.
Huang B, Lu M, Jia D, Ben-Jacob E, Levine H, Onuchic J N. Interrogating the topological robustness of gene regulatory circuits by randomization. PLoS Comput Biol. 2017 13(3):e1005456.
Iraia García-Santisteban, Godefridus J Peters, Elisa Giovannetti, Jose Antonio Rodríguez. USP1 deubiquitinase: cellular functions, regulatory mechanisms and emerging potential as target in cancer therapy. Mol Cancer. 2013; 12: 91.
Jung K Y, Wang H, Teriete P, Yap J L, Chen L, Lanning M E, Hu A, Lambert L J, Holien T, Sundan A, Cosford N D, Prochownik E V, Fletcher S. Perturbation of the c-Myc-Max protein-protein interaction via synthetic α-helix mimetics. J Med Chem. 2015 58(7):3002-24.
Kanteti R, Batra S K, Lennon F E, Salgia R. FAK and paxillin, two potential targets in pancreatic cancer. Oncotarget. 2016 7(21): 31586-31601.
Kawada I, Hasina R, Lennon F E, Bindokas V P, Usatyuk P, Tan Y H, Krishnaswamy S, Arif Q, Carey G, Hseu R D, Robinson M, Tretiakova M, Brand T M, lida M, Ferguson M K,
Wheeler D L, Husain A N, Natarajan V, Vokes E E, Singleton P A, Salgia R. Paxillin mutations affect focal adhesions and lead to altered mitochondrial dynamics: relevance to lung cancer. Cancer Biol Ther. 2013 14(7):679-91.
Kumar N, Cramer G M, Dahaj S A Z, Sundaram B, Celli J P, Kulkarni R V. Stochastic modeling of phenotypic switching and chemoresistance in cancer cell populations. Sci Rep. 2019 9(1):10845.
Liu S, Thomas S M, Woodside D G, Rose D M, Kiosses W B, Pfaff M, Ginsberg M H. Binding of paxillin to alpha4 integrins modifies integrin-dependent biological responses. Nature. 1999 402(6762):676-81.
Liu S, Ginsberg M H. Paxillin Binding to a Conserved Sequence Motif in the α4 Integrin Cytoplasmic Domain. J Biol Chem. 2000 275:22736-22742.
Lu M, Jolly M K, Levine H, Onuchic J N, Ben-Jacob E. MicroRNA-based regulation of epithelial-hybrid-mesenchymal fate determination. Proc Natl Acad Sci USA. 2013 110(45):18144-9
Ma X, Hammes S R. Paxillin actions in the nucleus. Steroids. 2018 133:87-92. Ma X, Biswas A, Hammes S R. Paxillin regulated genomic networks in prostate cancer. Steroids. 2019 Jul. 22; 151:108463.
Mackinnon A C, Tretiakova M, Henderson L, Mehta R G, Yan B C, Joseph L, Krausz T, Husain A N, Reid M E, Salgia R. Paxillin expression and amplification in early lung lesions of high-risk patients, lung adenocarcinoma and metastatic disease. J Clin Pathol. 2011 64(1):16-24.
Mahmoudabadi G, Rajagopalan K, Getzenberg R H, Hannenhalli S, Rangarajan G, Kulkarni P. Intrinsically disordered proteins and conformational noise: implications in cancer. Cell Cycle. 2013 12(1):26-31.
Mainiero F, Murgia C, Wary K K, Curatola A M, Pepe A, Blumemberg M, Westwick J K, Der C J, Giancotti F G. (1997). The coupling of α6β4 integrin to Ras-MAP kinase pathways mediated by Shc controls keratinocyte proliferation. EMBO Journal, 16(9), 2365-2375.
Mainiero F, Pepe A, Wary K K, Spinardi L, Mohammadi M, Schlessinger J, Giancotti F G. Signal transduction by the alpha 6 beta 4 integrin: distinct beta 4 subunit sites mediate recruitment of Shc/Grb2 and association with the cytoskeleton of hemidesmosomes. EMBO J. 1995 14(18):4470-81.
Marášek P, Dzijak R, Studenyak I, Fišerová J, Uličná L, Novák P, Hozák P. Paxillin-dependent regulation of IGF2 and H19 gene cluster expression. J Cell Sci. 2015 Aug. 15; 128(16):3106-16.
Martin-Yken H, François J M, Zerbib D. Knr4: a disordered hub protein at the heart of fungal cell wall signalling. Cell Microbiol. 2016 18(9):1217-27.
Maziveyi M, Alahari S K. Cell matrix adhesions in cancer: The proteins that form the glue. Oncotarget. 2017 8(29):48471-48487.
Neira J L, Bintz J, Arruebo M, Rizzuti B, Bonacci T, Vega S, Lanas A, Velázquez-Campoy A, lovanna J L, AbiAn O. Identification of a Drug Targeting an Intrinsically Disordered Protein Involved in Pancreatic Adenocarcinoma. Sci Rep. 2017 7:39732.
Patil A, Kinoshita K, Nakamura H. Hub promiscuity in protein-protein interaction networks. Int J Mol Sci. 2010 11(4):1930-43
Pisco A O, Brock A, Zhou J, Moor A, Mojtahedi M, Jackson D, Huang S. Non-Darwinian dynamics in therapy-induced cancer drug resistance. Nat Commun. 2013; 4:2467.
Qin Liang1, Thomas SDexheimer2, Ping Zhangl, Andrew SRosenthal2, Mark AVillamill, Changjun You3, Qiuting Zhang4, Junjun Chen1, Christine AOtt1, Hongmao Sun2, Diane K Luci2, Bifeng Yuan3, Anton Simeonov2, Ajit Jadhav2, Hui Xiao5, Yinsheng Wang3, David J Maloney2* & Zhihao Zhuangl*. A selective USP1-UAF1 inhibitor links deubiquitination to DNAdamage responses. Nat Chem Biol 2014 10 298-304.
Rocha C R R, Silva M M, Quinet A, Cabral-Neto J B, Menck C F M. DNA repair pathways and cisplatin resistance: an intimate relationship. Clinics (Sao Paulo). 2018 73(suppl 1):e478s.
Salgia R. Mutation testing for directing upfront targeted therapy and post-progression combination therapy strategies in lung adenocarcinoma. Expert Rev Mol Diagn. 2016 16(7):737-49.
Salgia R, Li J L, Lo S H, Brunkhorst B, Kansas G S, Sobhany E S, Sun Y, Pisick E, Hallek M, Ernst T, Ramana Tantravahi R, Lan Bo Chen, L B, James D. Griffin, J D. Molecular cloning of human paxillin, a focal adhesion protein phosphorylated by P210BCR/ABL. J Biol Chem. 1995 270(10):5039-47.
Sarin N, Engel F, Kalinda G O, Mannewitz M, Cinatl J Jr, Rothweiler F, Michaelis M, Saafan H, Ritter C A, Jaehde U, Frötschl R. Cisplatin resistance in non-small cell lung cancer cells is associated with an abrogation of cisplatin-induced G2/M cell cycle arrest. PLoS One. 2017 12(7):e0181081.
Schwartz N, Cohen R, Ben-Avraham D, Barabási A L, Havlin S. Percolation in directed scale-free networks. Phys Rev E Stat Nonlin Soft Matter Phys. 2002 66(1 Pt 2):015104.
Shaw L M, Rabinovitz I, Wang H H, Toker A, Mercurio A M. Activation of phosphoinositide 3-O H kinase by the alpha6beta4 integrin promotes carcinoma invasion. Cell. 1997 91(7):949-60.
Shen D W, Pouliot L M, Hall M D, Gottesman M M. Cisplatin resistance: a cellular self-defense mechanism resulting from multiple epigenetic and genetic changes. Pharmacol Rev. 2012 64(3):706-21.
Song J, Li M, Tretiakova M, Salgia R, Cagle P T, Husain A N. Expression patterns of PAX5, c-Met, and paxillin in neuroendocrine tumors of the lung. Arch Pathol Lab Med. 2010 134(11):1702-5.
Staby L, O'Shea C, Willemoës M, Theisen F, Kragelund B B, Skriver K. Eukaryotic transcription factors: paradigms of protein intrinsic disorder. Biochem J. 2017 474(15):2509-2532.
Su L, Lv X, Miao J. Integrin beta 4 in neural cells. Neuromolecular Med. 2008 10(4):316-21.
Swain, P. S., Elowitz, M. B. & Siggia, E. D. Intrinsic and extrinsic contributions to stochasticity in gene expression. Proc. Natl Acad. Sci. USA 2002 99:12795-12800.
Tan Y C, Mirzapoiazova T, Won B M, Zhu L, Srivastava M K, Vokes E E, Husain A N, Batra S K, Sharma S, Salgia R. Differential responsiveness of MET inhibition in non-small-cell lung cancer with altered CBL. Sci Rep. 2017 7(1):9192.
Tohru Yamamori, Hironobu Yasui, Masayuki Yamazumi, Yusuke Wada, Yoshinari Nakamura, Hideo Nakamura, Osamu Inanami. Ionizing radiation induces mitochondrial reactive oxygen species production accompanied by upregulation of mitochondrial electron transport chain function and mitochondrial content under control of the cell cycle checkpoint. Free Radical Biol Med 2012 53(2) 260-270.
Tözeren A, Kleinman H K, Wu S, Mercurio A M, Byers S W. Integrin alpha 6 beta 4 mediates dynamic interactions with laminin. J Cell Sci. 1994 November; 107(Pt 11): 3153-3163.
Turoverov K K, Kuznetsova I M, Fonin A V, Darling A L, Zaslavsky B Y, Uversky V N. Stochasticity of Biological Soft Matter: Emerging Concepts in Intrinsically Disordered Proteins and Biological Phase Separation. Trends Biochem Sci. 2019 44(8):716-728.
Trusolino L, Bertotti A, Comoglio P M. A signaling adapter function for alpha6beta4 integrin in the control of HGF-dependent invasive growth. Cell. 2001 107(5):643-54.
Tsafou K, Tiwari P B, Forman-Kay J D, Metallo S J, Toretsky J A. Targeting Intrinsically Disordered Transcription Factors: Changing the Paradigm. J Mol Biol. 2018 430(16):2321-2341.
Turner C E. Paxillin. Int J Biochem Cell Biol. 1998 30(9):955-9.
Turner C E. Paxillin and focal adhesion signalling. Nat Cell Biol. 2000 2(12):E231-6.
Wójcik S, Birol M, Rhoades E, Miranker A D, Levine Z A. Targeting the Intrinsically Disordered Proteome Using Small-Molecule Ligands. Methods Enzymol. 2018 611:703-734.
Wright P E, Dyson H J. Intrinsically disordered proteins in cellular signalling and regulation. Nat Rev Mol Cell Biol. 2015 16(1):18-29.
Wu D W, Wu T C, Wu J Y, Cheng Y W, Chen Y C, Lee M C, Chen C Y, Lee H. Phosphorylation of paxillin confers cisplatin resistance in non-small cell lung cancer via activating ERK-mediated Bcl-2 expression. Oncogene. 2014 33(35):4385-95.
Xiao L, Lan X, Shi X, Zhao K, Wang D, Wang X, Li F, Huang H, Liu J. Cytoplasmic RAP1 mediates cisplatin resistance of non-small cell lung cancer. Cell Death Dis. 2017 8(5):e2803.
Yao H, Jiang F, Lu A, Zhang G. Methods to Design and Synthesize Antibody-Drug Conjugates (ADCs). Int J Mol Sci. 2016 17(2). pii: E194.
Yu C, Niu X, Jin F, Liu Z, Jin C, Lai L. Structure-based Inhibitor Design for the Intrinsically Disordered Protein c-Myc. Sci Rep. 2016 6:22298.

1. A method of alleviating resistance to chemotherapy in a subject, comprising administering to the subject a composition comprising an siRNA that inhibits ITGB4 expression, an siRNA that inhibits PXN expression, or both that perturb the interaction between integrin β4 (ITGB4) and paxillin (PXN).

2.-4. (canceled)

5. The method of claim 1, wherein the subject suffers from one or more solid tumors.

6. The method of claim 1, wherein the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma.

7. The method of claim 1, wherein the subject has developed or is at an elevated risk of developing resistance to platinum-based chemotherapy.

8. The method of claim 1, wherein the subject has developed or is at an elevated risk of developing resistance to cisplatin or carboplatin.

9. A composition for alleviating resistance to chemotherapy in a subject, comprising an siRNA that inhibits ITGB4 expression, an siRNA that inhibits PXN expression, or both that perturb the interaction between integrin β34 (ITGB4) and paxillin (PXN).

10.-12. (canceled)

13. The composition of claim 9, wherein the subject suffers from one or more solid tumors.

14. The composition of claim 9, wherein the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma.

15. The composition of claim 9, wherein the subject has developed or is at an elevated risk of developing resistance to platinum-based chemotherapy.

16. The composition of claim 9, wherein the subject has developed or is at an elevated risk of developing resistance to cisplatin or carboplatin.

17. A method of treating cancer in a subject, comprising administering to the subject a chemotherapeutic agent, and a composition comprising an siRNA that inhibits ITGB4 expression, an siRNA that inhibits PXN expression, or both that perturb the interaction between integrin β4 (ITGB4) and paxillin (PXN).

18.-20. (canceled)

21. The method of claim 17, wherein the subject suffers from one or more solid tumors.

22. The method of claim 17, wherein the subject suffers from testicular cancer, ovarian cancer, cervical cancer, breast cancer, bladder cancer, head and neck cancer, esophageal cancer, lung cancer such as non-small cell lung cancer, mesothelioma, brain tumor and neuroblastoma.

23. The method of claim 17, wherein the subject has developed or is at an elevated risk of developing resistance to platinum-based chemotherapy.

24. The method of claim 17, wherein the subject has developed or is at an elevated risk of developing resistance to cisplatin or carboplatin.

25. The method of claim 17, wherein the chemotherapeutic agent is administered at a reduced dose comparing to the dose of the chemotherapeutic agent when administered alone.

26.-34. (canceled)
